(19) United States
(12) Patent Application Publication
(10) Pub. No.: US 2010/0120780 A1
(43) Pub. Date: May 13, 2010

(54) TREATMENTS FOR PREMATURE EJACULATION IN HUMANS

(76) Inventor: Chandra Ulagaraj Singh, San Antonio, TX (US)

Correspondence Address: REED SMITH LLP, 2500 ONE LIBERTY PLACE, 1650 MARKET STREET, PHILADELPHIA, PA, 19103, US

(21) Appl. No.: 12/450,866

(22) PCT Filed: Apr. 18, 2008
(86) PCT No.: PCT/US08/60874

§ 371 Date: Jan. 19, 2010

Related U.S. Application Data
(60) Provisional application No. 60/912,760, filed on Apr. 19, 2007.

(51) Int. Cl. A61K 31/165; A61K 31/439; A61K 31/445; A61K 31/451; A61K 31/46; A61K 31/519; A61K 31/4985; A61K 31/437; A61K 31/522; A61K 31/444; A61P 15/12

(52) U.S. Cl. 514/250; 514/627; 514/289; 514/317; 514/304; 514/252.16; 514/352; 514/299; 514/263.31; 514/334

(57) ABSTRACT

Provided are methods and compositions for the treatment of a sexual dysfunction such as premature ejaculation. In certain embodiments, a NMDA antagonist (e.g., dextromethorphan) is administered to a subject in combination with tramadol or a tramadol derivative to treat premature ejaculation. In certain embodiments, a capsaicinoid (e.g., capsaicin) and/or a phosphodiesterase type V inhibitor (e.g., sildenafil citrate) are further administered to the subject. Pharmaceutical preparations such as tablets and capsules are provided.

This application claims priority to U.S. Application No. 60/912,760 filed on Apr. 19, 2007, the entire disclosure of which is specifically incorporated herein by reference in its entirety without disclaimer.

### BACKGROUND OF THE INVENTION

1. Field of the Invention

The present invention relates generally to the fields of pharmaceutics and medicine. More particularly, it concerns pharmaceutical treatments for premature ejaculation.

2. Description of Related Art

Primitive premature ejaculation is regarded as the most common sexual disorder of the male. This may cause a loss of the ability to achieve sexual accommodation which is necessary for the satisfaction of the human instinctive desire. Recently, it has been determined that the number of cases manifesting various symptoms caused by such loss of sexual accommodation is rather large. The sexual problems due to premature ejaculation in men lead to social difficulties, such as asthenia due to the loss of self-confidence, as well as domestic discord. Premature ejaculation is defined as persistent or recurrent ejaculation before, upon, or shortly after penetration.

By nature, women typically experience the sex act markedly less intensely than a man at the commencement of sexual activity. Women thus typically require more time in order to reach the orgasm which provides natural relaxation of the whole nervous system strained to the maximum during the act. To this day the sense of touch plays an important role in human sex life; particularly sensitive to touch are the erogenous zones, first and foremost among them being the areas where skin borders on mucous membrane as, for example, in the vicinity of the oral cavity, the rectum, female genitals and breast nipples. The erogenous zone of a woman can be her entire body surface. In such cases it is possible to evoke lascivious feelings in her by touching any part of her body. But it is most often the case that erogenous zones are localized in strictly defined places such as: the clitoris, labia minora and the vagina. There are, additionally, many such sensitive points apart from the sex organs. These are: the lips, the ears, eyelids, neck, nipples, etc. In some cases these points are so sensitive that merely touching them can produce an orgasm in a woman.

However in the case of men, the erogenous zones are generally confined to the genitals and adjacent areas. At the commencement of the sex act the man already finds himself at a certain level of excitement, which is essential to erection and without which this act becomes quite impossible. Premature ejaculation generally prevents the continuation of sex out of consideration for the female because immediately after male orgasm and the associated ejaculation detumescence takes place and reduces or eliminates further frictiones in vagina.

A more preferable intercourse would be one in which, following immersing the penis into the vagina, both parties reached the boundary of orgasm simultaneously and, having crossed it, ended the sex act together (FIG. 1). This happens sometimes where a woman experienced in sexual intercourse can compensate for the excitement missing at the beginning of the act and reach the finishing line together with her partner in spite of that. For young and middle-aged men the norm of normal ejaculation vacillates between 2-6 minutes after the immersing the penis into the vagina.

Premature ejaculation occurs very frequently in the modern human sexual act. It concerns the fact that shortly after immersing the penis into the vagina takes place (FIG. 2), sometimes after 2-3 movements, ejaculation and orgasm occur; the erection vanishes and the sex act is ended. Obviously in such a situation the woman is only aroused, with little or no probability of orgasm during sex. Sexual satisfaction and normal relaxation of the female partner is typically affected or prevented in the presence of male impotence, whether through inadequate erection or through premature ejaculation.

Erection of the penis may be a self-perpetuating process of three steps: 1) vasodilation; 2) release of endogenous smooth-muscle relaxants; and, 3) progression of these effects distal from the initial site of onset. This has been termed the “cascade effect” (Andersson et al, 1995). Papaverine is an opium alkaloid and works as a smooth muscle relaxer possibly by cyclic GMP phosphodiesterase inhibition. It relaxes the musculature of the vascular system of the penis and increases blood flow (Papaverine Topical Gel Treatment For Erectile Dysfunction, Urology, Vol. 133(2); (1995), pp. 361-365). Another compound found useful in the treatment of impotence is prostaglandin E1, a naturally occurring compound that acts to increase arterial inflow to the penis and may also restrict venous outflow. Prostaglandin E1 is preferred to other compounds used in injections for the treatment of impotence because it is metabolized locally in the penis and is less likely to cause systemic symptoms such as hypotension. As a modified vascular tissue, corpora cavernosa of the penis (ccp) produces and secretes the same range of autocrine and paracrine regulators as conventional vascular tissue. The smooth muscle tone of the ccp, however, does not appear to be regulated in the same manner as in the vascular wall. Presently it is postulated that the tone or contractility of ccp is modulated by adrenergic regulation and locally produced NO and endothelin. In the ccp, most studies have been directed to observing the relaxing effects of NO (Rajfer et al 1992; Burnett 1995), vasoactive intestinal peptide (VIP), calcitonin gene-related peptide (CGRP) and parasympathetic innervation, which also have similar effects on conventional and ccp vascular smooth muscle.

During normal penile erections, when the inflow of blood to the ccp engages the sinusoidal spaces, the trabecular tissue compresses small cavernosal veins against the thick fibrous tissue surrounding the corpora to maintain the erection. To mediate these changes in blood flow, nitric oxide is released from postsynaptic parasympathetic neurons and, to a lesser extent, endothelial cells and α-adrenergic neurons are inhibited in the arterial and trabecular smooth muscle. Nitric oxide, which is readily diffusible, stimulates the formation of increased cyclic guanosine monophosphate (GMP) in the corpus cavernosum by guanylate cyclase to relax the smooth muscle cells.

Although effective for the treatment of erectile dysfunction, sildenafil has not shown to be effective in the treatment of premature ejaculation. Recently, the oral use of the citrate salt of sildenafil has been approved by the U.S. Food and Drug Administration (FDA) for the treatment of male erectile dysfunction. The composition of matter of sildenafil is described in the European patent EP 0463756. Sildenafil is reported to be a selective inhibitor of cyclic-GMP-specific phosphodiesterase type 5 (PDE5), the predominant isozyme metabolizing cyclic GMP formed in the corpus cavernosum (Boolell et al 1996). Since sildenafil is a potent inhibitor of PDE5 in the corpus cavernosum, it is believed to enhance the effect of nitric oxide, thereby increasing cavernosal blood flow in the penis, especially with sexual stimulation. Inasmuch as sildenafil at the currently recommended doses of 25-100 mg has little effect in the absence of sexual stimulation, sildenafil is believed to restore the natural erectile response to sexual stimulation but not cause erections in the absence of such stimulation (Goldstein 1998). The localized mechanism by which cyclic GMP stimulates relaxation of the smooth muscles has not been elucidated.

The oral use of the citrate salt of sildenafil has been approved for the treatment of male erectile dysfunction. Sildenafil is reported to be a selective inhibitor of cyclic-GMP-specific phosphodiesterase type 5 (PDE5), the predominant isozyme metabolizing cyclic GMP formed in the corpus cavernosum (Boolell et al 1996). Since sildenafil is a potent inhibitor of PDE5 in the corpus cavernosum, it is believed to enhance the effect of nitric oxide, thereby increasing cavernosal blood flow in the penis, especially with sexual stimulation. Inasmuch as sildenafil at the currently recommended doses of 25-100 mg has little effect in the absence of sexual stimulation, sildenafil is believed to restore the natural erectile response to sexual stimulation but not cause erections in the absence of such stimulation (Goldstein 1998). The localized mechanism by which cyclic GMP stimulates relaxation of the smooth muscles has not been elucidated.

Hull et al. (1994) observed that nitric oxide (NO) may inhibit seminal emission in male rats, probably by decreasing sympathetic nervous system activity. Kriegsfeld et al. (1999) noted that mice lacking endothelial NO synthase (eNOS) showed a higher incidence of premature ejaculation. In addition, Heuer et al. (2002) observed in vitro that the NO-cGMP cascade in part regulates human seminal vesicle contractility. Furthermore, it has been suggested that nitric oxide activity in the medial preoptic area tonically inhibits ejaculation by decreasing sympathetic tone (Pfaus 1999). These are rationales for using NO donating drugs as pharmacotherapy for PE. Sildenafil is a selective inhibitor of cyclic guanosine monophosphate (cGMP) specific phosphodiesterase type 5, which has been approved as a first line oral therapy for erectile dysfunction (Goldstein 1998; McMahon 2000). It thus enhances the relaxant effect of nitric oxide released in response to sexual stimulation by increasing cGMP concentrations in the corporal smooth muscle (Padma-Nathan 1999). In a study sildenafil administered as needed as a single treatment for PE, increased ejaculation time more than paroxetine (Abdel-Hamid 2001). In contrast, clomipramine, sertraline and paroxetine appear to be comparable in terms of efficacy. A number of studies suggest that adding a PDE5 inhibitor such as sildenafil to an SSRI such as paroxetine is better for PE than either drug alone (Abdel-Hamid 2004; Salonia 2002). Abdel-Hamid attributed the positive result associated with sildenafil use to following possible mechanisms. The first may be possible reduction in performance anxiety and the second is that sildenafil may maintain erection and increase the erection time, and ejaculation latency time was reported to be dependent on erection time.

Normal ejaculatory function in the human male implies a coordinated sequence of smooth and striate muscular contractions to promote projectile, antegrade transport of seminal fluid. This process begins with transmission of afferent nerve stimuli via the internal pudendal nerve from the penile shaft to higher centers. To complete the ejaculatory reflex efferent stimuli are transmitted from the anterolateral columns of the spinal cord and emerging from the thoracolumbar level to comprise a hypogastric or sympathetic plexus. From the interior mesenteric ganglion short adrenergic postganglionic fibers terminate in the seminal vesicles, vasal ampullae, and bladder neck. Sympathetic innervation of the seminal vesicles results in seminal emission into the posterior urethra. Appropriately timed bladder neck closure prevents retrograde passage of this semen bolus, which is propelled in the antegrade direction by clonic contracts of the bulbocavernosus and ischiocavernosus muscles of the pelvic floor. Ejaculation is a centrally, integrated peripheral evoked reflex, which occurs as a result of α1-adrenergic receptor activation. Effective pharmacological drugs for the treatment of premature ejaculation exist, but they suffer from severe side effects, for example clomipramine and phenoxybenzamine. Other treatments have a limited effectiveness (metoclopramide and the like).

At present, the treatment of choice for premature ejaculation is psychotherapy, either as a behavioral dual team sex therapy according to Master & Johnson protocol, or individual psychotherapy (Rifelli and Moro, Sessuologia Clinica. Bologna, (1989)). Previous methods of treating premature ejaculation include psychological therapies, topical anesthetics and the use of devices (U.S. Pat. Nos. 5,535,758, 5,063,915, 5,327,910, and 5,468,212). All of these methods may have significant drawbacks. Psychological therapies benefit only a subset of patients and require specialized therapists who may not be available to all patients, particularly in remote areas. Furthermore, psychological therapies cannot alleviate premature ejaculation resulting from non-psychological causes. Anesthetic agents decrease sensitivity of tissues, thereby diminishing sexual pleasure. Also, topical anesthetics can be transferred to sexual partners and thereby decrease their sensitivity and pleasure as well. With regard to devices, these can be awkward, inconvenient and embarrassing to use. Devices are highly conspicuous, and reveal the very condition which the suffering partner may prefer to conceal. Additionally, devices can cause irritation to one or both partners.

Methods for treating premature ejaculation by systemic administration of several different antidepressant compounds have been described (U.S. Pat. Nos. 4,507,323, 4,940,731, 5,151,448, and 5,276,042; PCT Publication No. WO95/13072). However, these drugs may not be effective for all patients, and the side effects of these drugs can halt treatment or impair patient compliance. Disease states or adverse interactions with other drugs may contraindicate the use of these compounds or require lower dosages that may not be effective to delay the onset of ejaculation. Additionally, the stigma of mental illness associated with antidepressant therapy can discourage patients from beginning or continuing such treatments. Administration of the antidepressant fluoxetine has been claimed to treat premature ejaculation (U.S. Pat. No. 5,151,448). However, the administration of fluoxetine may have many undesired aspects. Patients with hepatic or renal impairments may not be able to use fluoxetine due to its metabolism in the liver and excretion via the kidney. Systemic events during fluoxetine treatment involving the lungs, kidneys or liver have occurred, and death has occurred from overdoses. In addition, side effects of oral fluoxetine administration include hair loss, nausea, vomiting, dyspepsia, diarrhea, anorexia, anxiety, nervousness, insomnia, drowsiness, fatigue, headache, tremor, dizziness, convulsions, sweating, pruritis, and skin rashes. Fluoxetine interacts with a range of drugs, often by impairing their metabolism by the liver.

U.S. Pat. No. 4,940,731 describes the oral or parenteral administration of sertraline for treating premature ejaculation. It has been recognized that sertraline shares many of the same problems as fluoxetine; (see Martindale, The Extra Pharmacopoeia, 31st edition, at p. 333 (London: The Royal Pharmaceutical Society, 1996)). Sertraline is metabolized in the liver, and is excreted in the urine and feces. Thus, patients with cirrhosis must take lower doses, and caution must be exercised when administering sertraline to patients with renal impairment. Individuals taking monoamine oxidase inhibitors cannot take sertraline due to the risk of toxicity, leading to memory changes, confusion, irritability, chills, pyrexia and muscle rigidity. Side effects resulting from oral sertraline administration include nausea, diarrhea, dyspepsia, insomnia, somnolence, sweating, dry mouth, tremor and mania. Rare instances of coma, convulsions, fecal incontinence and gynecomastia have occurred in patients undergoing sertraline therapy. U.S. Pat. No. 5,276,042 describes the administration of paroxetine for the treatment of premature ejaculation. Paroxetine is predominantly excreted in the urine, and decreased doses are recommended in patients with hepatic and renal impairments. Like sertraline, paroxetine cannot be given to patients undergoing treatment with a monoamine oxidase inhibitor. Side effects from oral administration of paroxetine include hyponatremia, asthenia, sweating, nausea, decreased appetite, oropharynx disorder, somnolence, dizziness, insomnia, tremor, anxiety, impaired micturition, weakness and paresthesia. Thus there is a need for a method of treating premature ejaculation that requires no specialized psychological therapy, can be used conveniently and without embarrassment, and does not involve the problems associated with prior therapeutic methods.

U.S. Pat. No. 6,037,360 discloses that administration of various serotonin agonists and antagonists is effective in the treatment of premature ejaculation. The adverse effects occurring most frequently during treatment with serotonin inhibitors are gastrointestinal disturbances, such as, for example nausea, diarrhoea/loose stools, constipation. (Drugs 43 (Suppl. 2), 1992). Nausea is the main adverse effect in terms of incidence. Moreover it has been frequently observed that after administration of serotonin inhibitors, patients suffer from dyspepsia.

U.S. Pat. No. 5,707,999 teaches that two specific al-blockers, alfuzosine and terazosine, are effective in the treatment of psychogenic premature ejaculation and said drugs turned out to be effective in patients who proved to have no benefit from psychological therapy. However terazosine and its analogs have several side effects including headache, nausea, weight gain, dizziness, somnolence, dyspnea and blurred vision.

U.S. Pat. No. 6,037,346 discloses the local administration of phosphodiesterase inhibitors for the treatment of erectile dysfunction and a preferred mode of administration is claimed as transurethral. Pharmaceutical formulations and kits are provided as well. US application US 2002/0037828 A1 discloses the use of phosphodiesterase inhibitors for treating premature ejaculation.

U.S. Pat. Nos. 4,656,177 and 4,777,174 disclose combinations of non-narcotic analgesics/nonsteroidal anti-inflammatory drugs and/or narcotic analgesics and caffeine. The compositions elicit a more potent and more rapid analgesic response than if the pain reliever is given alone.

U.S. Pat. No. 5,248,678 teaches a method of increasing the arousal an alertness of comatose patients or near-comatose patients comprising administering to the patients effective amounts of an adenosine receptor antagonist, such as caffeine, and a GABA agonist, such as gabapentin.

PCT/US2006/61873, which is incorporated by reference in its entirety herein without disclaimer, describes a method for the treatment of premature ejaculation comprising administering a NMDA antagonist and a μ opiate receptor agonist. Unfortunately, certain NMDA antagonists, such as dextromethorphan which is also found in cough syrups, have potential for abuse if taken at inappropriately high doses. Thus, these compositions have the disadvantage of the possibility that an unscrupulous patient might try to achieve a dissociative hallucinogenic state by taking inappropriately high doses of the pharmaceutical compositions. This problem in modern medicine is not unique, and the abuse potential of many other pharmaceuticals, such as prescription pain medicines, have been widely documented and cited in the media. In view of the foregoing, there exists a clear need for improved treatments to treat premature ejaculation.

### SUMMARY OF THE INVENTION

The present invention overcomes limitations in the prior art by providing improved treatments for premature ejaculation. In particular, the compositions of the present invention include a capsaicinoid or an esterified capsaicinoid in the pharmaceutical preparations which comprise an NMDA antagonist and a μ opiate agonist, such as tramadol, and may be used to treat premature ejaculation. Capsaicinoids such as capsaicin are found in chilli peppers including jalapenos and habanero peppers. At higher concentrations, the capsaicinoid or esterified capsaicinoid can produce a burning sensation by stimulating villanoid receptors on neurons involved in pain perception. Thus, the pharmaceutical preparations of the present invention reduce the abuse potential of the compositions of the present invention by providing a strong deterrent at higher concentrations. Additionally, it has been discovered that administration of a capsaicinoid or esterified capsaicinoid such as capsaicin palmitate to a subject can result in additional therapeutic benefit for the treatment of premature ejaculation. Without wishing to be bound by any theory, it is believed that the action of the capsaicin at villanoid receptors and/or the analgesic effect that can occur at the spinal cord as a result of stimulation of specific neurons involved in pain perception may be responsible for the therapeutic effect for the treatment of premature ejaculation.

In certain embodiments, a combination of (1) a non-toxic NMDA receptor antagonist, such as dextromethorphan, with (2) tramadol or a derivative or analog of tramadol, and (3) a capsaicinoid or an esterified capsaicinoid can be used to treat premature ejaculation. It has also been discovered that a combination of (1) a non-toxic NMDA receptor antagonist such as dextromethorphan, (2) tramadol or a derivative or analog of tramadol, (3) a capsaicinoid or an esterified capsaicinoid and (4) a cyclic-GMP-specific phosphodiesterase type 5 (PDE5) inhibitor such as sildenafil exhibit significant palliative effects on premature ejaculation in individuals who suffer from both erectile dysfunction and premature ejaculation. To the knowledge of the inventor, there has been no recognition or appreciation that these combinations can be used to effectively to treat premature ejaculation in humans.

In one aspect, the present invention provides a method of effectively treating a sexual dysfunction in humans or other mammals. The method comprises administering to a patient in need of such treatment an amount of agents including a) an NMDA receptor antagonist or a pharmaceutically acceptable salt thereof, b) tramadol or a derivative or analog of tramadol, or a pharmaceutically acceptable salt thereof and optionally c) a capsaicinoid or an esterified capsaicinoid. The combined amount of agents may be used to effectively treat the sexual dysfunction.

In accordance with the present invention, the sexual dysfunction can be premature ejaculation or a sexual dysfunction that includes premature ejaculation as a component of the condition.

The agents can be administered separately or in combination. When three or more agents are involved, the agents can be administered in various combinations. For example, three agents can be administered together, or two of the agents can be administered together, while the third agent is administered separately. For example, the agents may be subsequently administered to the patient within a time period of from about 1 second to about 2 hours. The agents may be administered in a single pharmaceutical composition such as, e.g., a tablet or capsule.

The agents are preferably administered prior to sexual activity. Administration can be orally, by means of an implant, parenterally, sub-dermally, sublingually, rectally, topically, or via inhalation. In preferred embodiments, the agents are administered orally.

The NMDA receptor antagonist can be dextromethorphan, dextrorphan, ketamine, amantadine, memantine, eliprodil, ifenprodil, phencyclidine, MK-801, dizocilpine, CCPcnc, flupirtine, or derivatives or salts thereof. Preferably, the antagonist is dextromethorphan.

Tramadol or a derivative or analog of tramadol may also be administered to a patient or included in a pharmaceutical composition according to the present invention. Derivatives of tramadol which may be used with the present invention include (1R,2R or 1S,2S)-(dimethylaminomethyl)-1-(3-methoxyphenyl)-cyclohexanol (tramadol), its N-oxide derivative (“tramadol N-oxide”), its O-desmethyl derivative (“O-desmethyl tramadol”), venlafaxine, (R/S)-1-[2-(dimethylamino)-1-(4-methoxyphenyl)ethyl]cyclohexanol and O-desmethylvenlafaxine or mixtures, and stereoisomers or recemates thereof.

The agents can be administered in a dosage form as a tablet, a multiparticulate formulation for oral administration; a solution, a sustained release formulation, a suspension or elixir for oral administration, an injectable formulation, an implantable device, a topical preparation, a solid state and/or depot type transdermal delivery device(s), a suppository, a buccal tablet, or an inhalation formulation such as a controlled release particle formulation or spray, mist or other topical vehicle, intended to be inhaled or instilled into the sinuses. The dosage form can be further defined as a solid oral dosage form formulated as a tablet or capsule.

In accordance with the present invention, the ratio of NMDA receptor antagonist to tramadol or a derivative or analog of tramadol can be from about 15:1 to 1:15, about 10:1 to 1:10, about 5:1 to 1:5, or about 1:2.

In certain embodiments of the present invention, a phosphodiesterase (PDE) inhibitor or a pharmaceutically acceptable salt thereof is included as one of the agents. Preferably, the PDE inhibitor is a phosphodiesterase type 5 (EC 3.1.4.17) inhibitor. The PDE inhibitor can be sildenafil, vardenafil, tadalafil, aminophylline, theophylline, aminone, milrinone, vesnarinone, vinpocetine, pemobendan, cilostamide, enoximone, peroximone, rolipram, R020-1724, zaniprast, dipyridamole, MY5445, IC-351, or a pharmaceutically acceptable salt thereof. The ratio of NMDA receptor antagonist to phosphodiesterase inhibitor to tramadol or a derivative or analog of tramadol can be from about 90:1:1 to 1:90:1 to 1:1:90. The ratio by weight of NMDA receptor antagonist to capsaicinoid or esterified capsaicinoid to the tramadol or the derivative or analog of tramadol may be from about 90:1:1 to 1:90:1 to 1:1:90.

In certain embodiments, the capsaicinoid may be selected from the group consisting of capsaicin, capsaicin palmitate, civamide, homocapsaicin, nordihydrocapsaicin, dihydrocapsaicin, homodihydrocapsaicin, n-vanillyloctanamide, nonivamide and n-vanillyldecanamide. The esterified capsaicin may be of formula (I):

R—CO—CAP (I)

wherein CAP is capsaicin, a capsaicin analogue, civamide, homocapsaicin, nordihydrocapsaicin, dihydrocapsaicin, homodihydrocapsaicin, n-vanillyloctanamide, nonivamide or n-vanillyldecanamide; R may be a C₁-C₁₈ alkyl group, a C₁-C₁₈ aryl group, a C₁-C₁₈ alkylene group, a C₁-C₁₈ arylene group, —CH₂—CH₂—COOH or a c-pentenyl group. R may be selected from the group consisting of methyl, ethyl, propyl, butyl, hexyl, heptyl, octyl, dodecyl, 1-pentadecyl, 1-heptadecyl, isopropyl, sec-butyl, t-butyl, 2-methylbutyl, 2-pentyl, 3-pentyl, cyclopropyl, cyclobutyl, cyclopentyl and cyclohexyl, vinyl (ethenyl), 1-propenyl, i-butenyl, pentenyl, hexenyl, n-decenyl, —CH₂—CH₂—COOH and c-pentenyl groups. In certain embodiments, the pharmaceutical composition comprises capsaicin and/or capsaicin palmitate.

In another aspect, the present invention provides a pharmaceutical composition comprising a therapeutically effective amount of a combination of agents. The combination may comprise a) an NMDA receptor antagonist or a pharmaceutically acceptable salt thereof, b) tramadol or a derivative or analog of tramadol, or a pharmaceutically acceptable salt thereof, c) capsaicin or an ester of capsaicin and d) a phosphodiesterase type V inhibitor or a pharmaceutically acceptable salt thereof.

In accordance with the present invention, compositions (e.g., including combinations of pharmacologically active compounds and formulations thereof) have been developed which can be administered to a human in the treatment of premature ejaculation. These compositions may include non-toxic dosage amounts of a drug, such as, for example, tramadol or a derivative or analog of tramadol, an effective non-toxic dosage amount of an NMDA receptor antagonist such as dextromethorphan (e.g., dextromethorphan hydrate or a salt thereof), a PDE5 inhibitor (e.g., sildenafil) and an effective non-toxic dosage amount of capsaicin or an ester of capsaicin.

The compositions may be administered to a human in the treatment of premature ejaculation and erection. These compositions may include a plurality of effective non-toxic dosage amounts of a drug which inhibits cyclic-GMP-specific phosphodiesterase type 5 (PDE5), for example, sildenafil (or salt thereof), an effective non-toxic dosage amount of an NMDA receptor antagonist such as dextromethorphan (preferably dextromethorphan hydrate or salt thereof), an effective non-toxic dosage amount of tramadol or a derivative or analog of tramadol, for example, tramadol (or salt thereof) and a capsaicinoid or an esterified capsaicinoid.

According to yet another aspect of the invention, applicant has provided pharmaceutical compositions comprising a plurality of dosage amounts each comprising, together with pharmaceutical excipients suitable for oral or parenteral administration, a therapeutically effective amount of agents. The amount may be effective to treat and to assist to resolve diseases and conditions of premature ejaculation in the human male in a manner that is essentially or completely non-toxic to the patient. The therapeutically effective dosage amount of agents may include tramadol or a derivative or analog of tramadol and an effective non-toxic dosage amount of an NMDA receptor antagonist such as dextromethorphan and/or salts thereof (for example the hydrobromide or hydrochloride salt) and/or homologues, analogues, derivatives, complexes, prodrugs, esters, and/or fragments thereof, and an effective non-toxic dosage amount of a capsaicinoid or an esterified capsaicinoid.

It is another aspect of the invention to provide a method wherein each pharmacologically active agent is administered orally. It is a further aspect of the invention to provide a method wherein each pharmacologically active agent is administered parenterally.

In accordance with the invention, pharmaceutical formulations are provided for carrying out the method of the invention. The pharmaceutical formulations may comprise an effective amount of a selected tramadol or a derivative or analog of tramadol, an NMDA receptor antagonist such as dextromethorphan, a capsaicinoid or an esterified capsaicinoid, a pharmacologically acceptable carrier or vehicle, and, optionally (i.e., in topical, transdermal or transurethral formulations), an enhancer. Other types of components may be incorporated into the formulation as well, e.g., excipients, surfactants, preservatives (e.g., antioxidants), stabilizers, enzyme inhibitors, chelating agents, and the like, as will be appreciated by those skilled in the art of pharmaceutical formulation preparation and drug delivery.

Yet another aspect of the subject invention is the disclosure that a combination of cyclic-GMP-specific phosphodiesterase type 5 (PDE5) inhibitors such as sildenafil which can facilitate the erection of the penis in humans under sexual stimulation, an NMDA receptor antagonist such as dextromethorphan which involves in anti-excitotoxic activity in humans, tramadol or a derivative or analog of tramadol, and a capsaicinoid or an esterified capsaicinoid, is very effective in delaying the onset of ejaculation in male humans who have erection as well as ejaculation problems.

The pharmaceutical compositions may comprise a plurality of dosage amounts one or more: (1) pharmaceutical excipients suitable for oral or parenteral administration, (2) a therapeutically effective amount (e.g., sufficient to treat, assist, or resolve premature ejaculation in a human male, preferably at dosages that are essentially or completely non-toxic to the patient) of a drug for example which inhibits cyclic-GMP-specific phosphodiesterase type 5 (PDE5) such as, for example, sildenafil, (3) a therapeutically effective amount of an NMDA receptor antagonist such as dextromethorphan, and (4) an effective non-toxic dosage amount of tramadol or a derivative or analog of tramadol. In various embodiments, derivatives of dextromethorphan and/or tramadol may be substituted for or used in combination with the foregoing pharmaceutical composition. Derivatives of dextromethorphan and tramadol include homologues, analogues, derivatives, complexes, prodrugs, esters, and pharmacologically active fragments thereof. As would be appreciated by one of skill, a pharmacologically acceptable salts of dextromethorphan and/or tramadol, such as the hydrobromide salts may be included in the pharmacological preparations described herein. Drug delivery may be accomplished through any route effective to provide relief from premature ejaculation, including oral, parenteral, buccal, rectal, topical, transdermal, transurethral, and intracavernosal injection.

As with compositions containing a cytochrome P450 inhibitor, compositions containing a PDE5 inhibitor can also comprise a pharmacologically acceptable carrier or vehicle, and, optionally (i.e., in topical, transdermal or transurethral formulations), an enhancer. Other types of components may be incorporated into the formulation as well, e.g., excipients, surfactants, preservatives (e.g., antioxidants), stabilizers, enzyme inhibitors, chelating agents, and the like, as will be appreciated by those skilled in the art of pharmaceutical formulation preparation and drug delivery.

The NMDA antagonist and/or at least one pharmaceutically acceptable salt thereof can be administered before, simultaneously with, or after administration of the other neuroactive agents such as tramadol or other μ-opiate agonist or agonist/antagonist or salt thereof. The dosing interval of administration of the NMDA antagonist may overlap or be administered simultaneously or in the same pharmaceutical preparation with tramadol and/or other μ-opiate agonist or agonist/antagonist.

Also, the cyclic-GMP-specific phosphodiesterase type 5 (PDE5) inhibitor and/or at least one pharmaceutically acceptable salt thereof can be administered before, simultaneously with, or after administration of the tramadol or other μ-opiate agonist or agonist/antagonist and/or at least one pharmaceutically acceptable salt thereof and the NMDA antagonist and/or at least one pharmaceutically acceptable salt thereof, such that the dosing interval of the cyclic-GMP-specific phosphodiesterase type 5 (PDE5) inhibitor and/or at least one pharmaceutically acceptable salt thereof overlaps with the dosing interval of the tramadol or other μ-opiate agonist or agonist/antagonist and/or at least one pharmaceutically acceptable salt thereof and the dosing interval of the NMDA antagonist and/or at least one pharmaceutically acceptable salt thereof. Capsaicin or an ester of capsaicin may also be administered prior to, during, or after the administration of a PDE5 inhibitor, a g-opiate antagonist to treat premature ejaculation.

Additional objects, advantages and novel features of the invention will be set forth in part in the description which follows, and in part will become apparent to those skilled in the art upon examination of the following, or may be learned by practice of the invention.

Before describing the present invention in detail, it is to be understood that this invention is not limited to particular drugs or drug delivery systems, as such may vary. It is also to be understood that the terminology used herein is for the purpose of describing particular embodiments only, and is not intended to be limiting.

As used in this specification, the singular forms “a,” “an” and “the” include plural referents unless the context clearly dictates otherwise. Thus, for example, reference to “a pharmacologically active agent” includes a combination of two or more pharmacologically active agents, and the like. In describing the present invention, the following terminology will be used in accordance with the definitions set out below.

The terms “active agent,” “drug” and “pharmacologically active agent” are used interchangeably herein to refer to a chemical material or compound which, when administered to an organism (human or animal) induces a desired pharmacologic effect. Included are derivatives and analogs of those compounds or classes of compounds specifically mentioned which also induce the desired pharmacologic effect.

The term “topical administration” is used in its conventional sense to mean delivery of a topical drug or pharmacologically active agent to the skin or mucosa.

“Carriers” or “vehicles” as used herein refer to carrier materials suitable for drug administration. Carriers and vehicles useful herein include any such materials known in the art, e.g., any liquid, gel, solvent, liquid diluent, solubilizer, or the like, which is nontoxic and which does not interact with other components of the composition in a deleterious manner.

By an “effective” amount of a drug or pharmacologically active agent is meant a nontoxic but sufficient amount of the drug or agent to provide the desired effect.

The term “premature ejaculation” as used herein intends a sexual dysfunction wherein a male is unable to control the ejaculatory process to a degree sufficient to satisfy a partner or ejaculates more quickly than desired. Generally, “premature ejaculation” refers to persistent or recurring ejaculation with minimal stimulation before or during sexual intercourse. The term includes both “congenital or lifelong” premature ejaculation and “primary or acquired” premature ejaculation as set forth, for example, in U.S. Pat. No. 5,151,448 and in Male Infertility and Sexual Dysfunction at p. 356 (New York: Springer-Verlag, 1997). See also Diagnostic and Statistical Manual of Mental Disorders (Washington, D.C.: American Psychiatric Association, 1994).

The term “NSAID” refers to non-steroidal substances which inhibit the production of prostaglandins by binding with cyclo-oxygenase enzymes. The compound acetaminophen is included under this category even though acetaminophen does not have anti-inflammatory properties but bind with cyclo-oxygenase enzymes in the periphery and at the hypothalamic thermoregulatory center.

The term “tramadol or a derivative or analog of tramadol” refers to any one of (1R,2R or 1S,2S)-(dimethylaminomethyl)-1-(3-methoxyphenyl)-cyclohexanol (tramadol), its N-oxide derivative (“tramadol N-oxide”), its O-desmethyl derivative (“O-desmethyl tramadol”), venlafaxine, (R/S)-1-[2-(dimethylamino)-1-(4-methoxyphenyl)ethyl]cyclohexanol and O-desmethylvenlafaxine or mixtures, stereoisomers or recemates thereof.

The term “sildenafil” as used herein includes the free base form of this compound as well as pharmacologically acceptable acid addition salts thereof formed with organo-carboxylic acids, organo-sulphonic acids or inorganic acids. For purposes of the present invention, the organo-carboxylic acid salt, sildenafil citrate, having a solubility in water of 3.5 mg/ml is particularly preferred. Reference to “sildenafil” includes sildenafil citrate.

The term “capsaicinoid” and “capsaicinoids,” as used herein, encompasses not only the compound capsaicin, but also homocapsaicin, nordihydrocapsaicin, dihydrocapsaicin, homodihydrocapsaicin and/or any compounded mixture thereof (see, e.g., FIG. 4).

### BRIEF DESCRIPTION OF THE DRAWINGS

FIG. 1: A graph showing orgasm levels during normal sexual intercourse. Orgasm levels in a man and woman during normal sexual intercourse are shown. The orgasm level is an arbitrary quantity describing the physical and emotional excitements during sexual intercourse.

FIG. 2: A graph showing orgasm levels in the case of premature ejaculation. The orgasm levels in male and female in the case of pre-mature ejaculation are shown. The orgasm level is an arbitrary quantity describing the physical and emotional excitements during sexual intercourse.

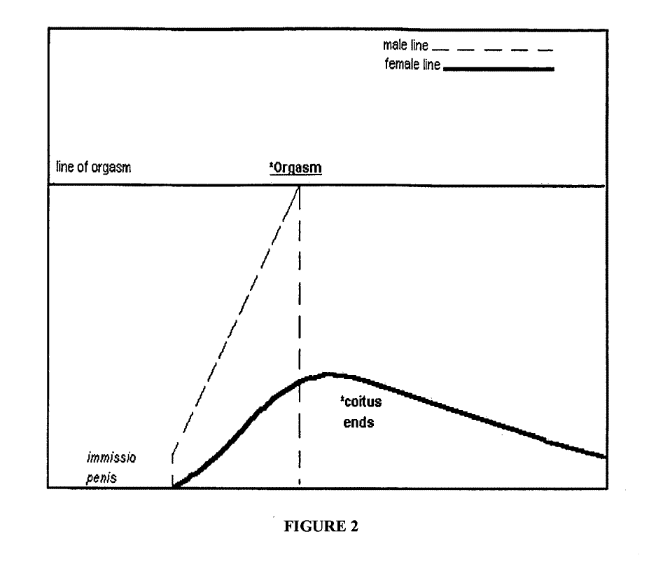

FIG. 3: The chemical structures of tramadol, dextromethorphan and sildenafil.

FIG. 4: The chemical structures of various capsaicinoids.

FIG. 5: Chemical structures of certain capsaicin esters.

### DESCRIPTION OF ILLUSTRATIVE EMBODIMENTS

The present invention overcomes limitations in the prior art by providing new treatments for premature ejaculation. In particular, it has been found that a combination of a non-toxic NMDA receptor antagonist, such as dextromethorphan, with tramadol or a derivative or analog of tramadol, and optionally a capsaicinoid and/or sildenafil are particularly effective for treating premature ejaculation.

Two distinct categories of premature ejaculation exist (CLASS I and CLASS II) and may be treated according to the present invention. For males who do not have any erection problem and to have satisfactory sexual intercourse but who nonetheless suffer from premature ejaculation (hereinafter referred to as “CLASS I”), the ejaculation process has to be delayed so that the sexual partners would have sufficient time for intercourse to reach maximum sexual satisfaction.

“CLASS II” males both have an erection problem and cannot control ejaculation once erection is achieved. For class II males, the following two steps are preferably followed: (1) the erection has to be achieved through certain pharmaceutical agents such as sildenafil such that the male will have full erection upon the stimulation by the sexual partner; (2) the ejaculation process has to be delayed so that the sexual partners would have sufficient time for intercourse to reach maximum sexual satisfaction.

Without wishing to be bound by any theory, administration of dextromethorphan (DM) causes an anti-excitotoxic effect in humans which can effect the ejaculation process. Tramadol has an analgesic effect due to its effect on the nerve signals. Administration of a debrisoquin hydroxylase inhibitor or a cytochrome-P450 inhibitor concurrently with DM can substantially increase the observable therapeutic effects of DM in human clinical trials (Pantich 2006). Here, the effectiveness of DM as an agent for treating premature ejaculation may also be increased by the co-administration of a cytochrome oxidase inhibitor. As shown in the below examples, administration of a combination of these agents can have a therapeutic effect and/or effectively treat premature ejaculation for CLASS I males. To his surprise, it has been discovered that ingestion of these agents can have profound effects on premature ejaculation and prolong the sexual intercourse to reach a substantially improved orgasm. Further it has been determined that these agents can be used to have multiple orgasm during sexual intercourse.

In addition, the inventor has discovered that ingestion of sildenafil, tramadol and DM has profound effects on the premature ejaculation in CLASS II males and that they prolong the sexual intercourse to reach maximal orgasm. Further he observed that these agents can be used to have multiple orgasm during sexual intercourse. Further the inventor has discovered that ingestion of tramadol along with sildenafil and DM does not substantially inhibit or antagonize the therapeutic effect of sildenafil. Thus sildenafil and DM may be administered in combination for treating premature ejaculation in CLASS II patients.

Additionally, in certain embodiments of the present invention, the addition of caffeine to a composition of the present invention can advantageously offset any drowsiness or sedation resulting from the opiate analgesic.

### I. TREATMENTS FOR PREMATURE EJACULATION IN CLASS I MALES

In order to carry out the method of the invention to treat premature ejaculation in CLASS I males, selected pharmacologically active agent(s) is administered to an individual. The active agents may be administered orally, parenterally, buccally, rectally, or locally by intracavernosal injection or by delivery to the urethra. In various embodiments, a μ-opiate anagesic such as tramadol, or active metabolites and/or salts thereof may be administered in combination with an NMDA antagonist such as tramadol.

A. Tramadol and Venlafaxine

(+/−)-Tramadol is a synthetic 4-phenyl-piperidine analogue of codeine (Shipton 2000). It is a central analgesic with a low affinity for opiate receptors. Its selectivity for μ-receptors has recently been demonstrated, and the M1 metabolite of tramadol, produced by liver O-demethylation, shows a higher affinity for opiate receptors than the parent drug. The rate of production of this M1 derivative (O-demethyl tramadol), is influenced by a polymorphic isoenzyme of the debrisoquine-type, cytochrome P450-2D6 (CYP2D6). One mechanism relates to its weak affinity for μ-opiate receptors (6,000-fold less than morphine, 100-fold less than d-propoxyphene, 10-fold less than codeine, and equivalent to dextromethorphan). Moreover, and in contrast to other opiates, the analgesic action of tramadol is only partially inhibited by the opiate antagonist naloxone, which suggests the existence of another mechanism of action. This was demonstrated by the discovery of a monoaminergic activity that inhibits noradrenaline (norepinephrine) and serotonin (5-hydroxytryptamine; 5-HT) reuptake, making a significant contribution to the analgesic action by blocking nociceptive impulses at the spinal level (Dayer et al. 1994 & 1997).

(+/−)-Tramadol is a racemic mixture of 2 enantiomers, each one displaying differing affinities for various receptors. (+/−)-tramadol is a selective agonist of μ receptors and preferentially inhibits serotonin reuptake, whereas (−)-tramadol mainly inhibits noradrenaline reuptake. The action of these 2 enantiomers is both complementary and synergistic and results in the analgesic effect of (+/−)-tramadol. After oral administration, tramadol demonstrates 68% bioavailability, with peak serum concentrations reached within 2 hours. The elimination kinetics can be described as 2-compartmental, with a half-life of 5.1 hours for tramadol and 9 hours for the M1 derivative after a single oral dose of 100 mg. This explains the approximately 2-fold accumulation of the parent drug and its M1 derivative that is observed during multiple dose treatment with tramadol. The recommended daily dose of tramadol is between 50 and 100 mg every 4 to 6 hours, with a maximum dose of 400 mg/day. The duration of the analgesic effect after a single oral dose of tramadol 100 mg is about 6 hours. Adverse effects, and nausea in particular, are dose dependent and therefore considerably more likely to appear if the loading dose is high. The reduction of this dose during the first days of treatment is an important factor in improving tolerability. Other adverse effects are generally similar to those of opiates, although they are usually less severe, and can include respiratory depression, dysphoria and constipation. Tramadol can be administered concomitantly with other analgesics, particularly those with peripheral action, while drugs that depress CNS function may enhance the sedative effect of tramadol. Tramadol has pharmacodynamic and pharmacokinetic properties that are highly unlikely to lead to dependence. This was confirmed by various controlled studies and postmarketing surveillance studies, which reported an extremely small number of patients developing tolerance or instances of tramadol abuse (Raffia et el, 1993; Lee et al, 1993). Although it has proven to be a safe and effective agent for the control of pain, adverse effects can occur with its use. It has been reported the occurrence of seizure activity after the inadvertent administration of 4 mg/kg of tramadol to a child (Tobias 1997).

Tramadol has the chemical name (+/−)-trans (RR,SS)-2-[(di-methylamino)methyl]-1-(3-methoxyphenyl)cyclohexanol, and which is often erroneously referred to in literature as the cis(RS,SR) diastereomer. Tramadol is a centrally acting, binary analgesic that is neither opiate-derived, nor is it an NSAID. It is used to control moderate pain in chronic pain settings, such as osteoarthritis and post-operative analgesia, and acute pain, such as dental pain.

Tramadol is a racemate and consists of equal quantities of (+)- and (−)-enantiomers. It is known that the pure enantiomers of tramadol have a differing pharmaceutical profiles and effects when compared to the racemate. The (+)-enantiomer is distinguished by an opiate-like analgesic action due its binding with the μ-opiate receptor, and both enantiomers inhibit 5-hydroxytryptamine (serotonin) and noradrenaline (norepinephrine) reuptake, which is stronger than that of racemic mixtures of tramadol, while distinct inhibition of noradrenaline reuptake is observed with the (−)-enantiomer. It has been proven for (+)- and (−)-tramadol that, depending upon the model, the two enantiomers mutually reinforce and enhance their individual actions (Raffa et al, 1993; Grond et al, 1995 and Wiebalck et al, 1998). The potent analgesic action of tramadol is likely based on this mutually dependent reinforcement of action of the enantiomers. Tramadol's major active metabolite, O-desmethyltramadol (M1), shows higher affinity for the μ-opiate receptor and has at least twice the analgesic potency of the parent drug. O-desmethyl-N-mono-desmethyltramadol (referred to as M5 in some places in the following text and in the literature) is known as one of the in vivo metabolites of tramadol (1RS, 2RS)-2[(dimethylamino)methyl]-1-(3-methoxyphenyl)cyclohexanol (Lintz et al, 1981). M5 penetrates the blood-brain barrier to only a limited extent, as the effects on the central nervous system, for example analgesic effects, are distinctly less pronounced on intravenous administration than on intracerebroventricular administration. Despite the fact that tramadol is chemically unrelated to the opiates adverse side effects associated with administration of tramadol are similar to those of the opiates if used at higher doses.

A non-limiting list of tramadol and derivatives of tramadol which may be utilized in the present invention include any one of (1R,2R or 1S,2S)-(dimethylaminomethyl)-1-(3-methoxyphenyl)-cyclohexanol (tramadol), its N-oxide derivative (“tramadol N-oxide”), its O-desmethyl derivative (“O-desmethyl tramadol”), venlafaxine, (R/S)-1-[2-(dimethylamino)-1-(4-methoxyphenypethyl]cyclohexanol and O-desmethylvenlafaxine or mixtures, stereoisomers, recemates, metabolites, salts or complexes thereof.

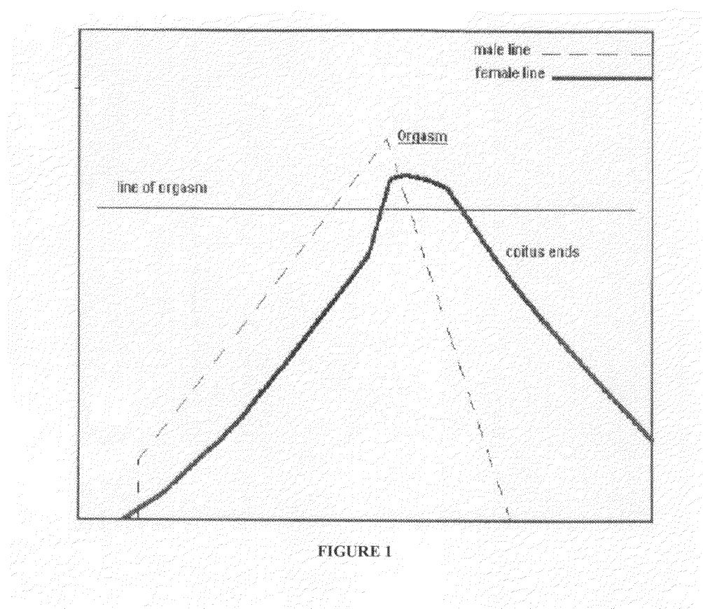

In various embodiments, venlafaxine may be substituted for or used in combination with tramadol. Venlafaxine is a novel SSRI chemically unrelated to other SSRIs but chemically similar to the tramadol (FIG. 1; Markowitz 1998). The chemical structures of venlafaxine and tramadol are similar, demonstrating the similarity between these two antidepressant and analgesic substances, respectively. It is designated (R/S)-1-[2-(dimethylamino)-1-(4-methoxyphenyl)ethyl]cyclohexanol or (±)-1-[a-[(dimethylamino)methyl]-p-methoxybenzyl]cyclohexanol and has the empirical formula of C₁₇H₂₇NO₂. Venlafaxine hydrochloride is a white to off-white crystalline solid with a solubility of 572 mg/mL in water (adjusted to ionic strength of 0.2 M with sodium chloride. Its octanol:water (0.2M sodium chloride) partition coefficient is 0.43. Venlafaxine hydrochloride (Effexor) is formulated as capsule for oral administration. Capsules contain venlafaxine hydrochloride equivalent to 37.5 mg, 75 mg, or 150 mg venlafaxine.

The mechanism of the antidepressant action of venlafaxine in humans is believed to be the same as with other SSRIs, associated with its potentiation of neurotransmitter activity in the CNS as with other SSRIs: preclinical studies have shown that venlafaxine and its active metabolite, O-desmethylvenlafaxine (ODV), are potent inhibitors of neuronal serotonin and norepinephrine reuptake and weak inhibitors of dopamine reuptake. That venlafaxine is analgesia is seen in studies in animals that show that venlafaxine is effective in reversing chronic neuropathic pain secondary to thermal hyperalgesia, and additionally is effective in treating the hyperalgesia of neuropathic pain due to chronic sciatic nerve constriction injury in rats (Lang 1998). Venlafaxine-induced antinociception is significantly inhibited by naloxone, nor-BNI and naltrindole but not by β-FNA or naloxonazine, implying involvement of κ1- and δ-opioid mechanisms. When adrenergic and serotoninergic antagonists are used, yohimbine but not phentolamine or metergoline, decreased antinociception elicited by venlafaxine, implying a clear α2- and a minor α1-adrenergic mechanism of antinociception. Therefore, the antinociceptive effect of venlafaxine is mainly influenced by the κ- and δ-opioid receptor subtypes combined with the α2-adrenergic receptor. These results suggest a potential use of venlafaxine in the management of some pain syndromes. However, further research may be needed in order to establish both the exact clinical indications and the effective doses of venlafaxine when prescribed for neuropathic pain (Schreiber 1999).

Venlafaxine is a novel SSRI chemically unrelated to other SSRIs but chemically similar to the tramadol (FIG. 1; Markowitz 1998). The chemical structures of venlafaxine and tramadol are similar, demonstrating the similarity between these two antidepressant and analgesic substances, respectively. It is designated (R/S)-1-[2-(dimethylamino)-1-(4-methoxyphenyl)ethyl]cyclohexanol or (±)-1-[a-[(dimethylamino)methyl]-p-methoxybenzyl]cyclohexanol and has the empirical formula of C₁₇H₂₇NO₂. Venlafaxine hydrochloride is a white to off-white crystalline solid with a solubility of 572 mg/mL in water (adjusted to ionic strength of 0.2 M with sodium chloride. Its octanol:water (0.2M sodium chloride) partition coefficient is 0.43. Venlafaxine hydrochloride (Effexor) is formulated as capsule for oral administration. Capsules contain venlafaxine hydrochloride equivalent to 37.5 mg, 75 mg, or 150 mg venlafaxine.

The mechanism of the antidepressant action of venlafaxine in humans is believed to be the same as with other SSRIs, associated with its potentiation of neurotransmitter activity in the CNS as with other SSRIs: preclinical studies have shown that venlafaxine and its active metabolite, O-desmethylvenlafaxine (ODV), are potent inhibitors of neuronal serotonin and norepinephrine reuptake and weak inhibitors of dopamine reuptake. That venlafaxine is analgesia is seen in studies in animals that show that venlafaxine is effective in reversing chronic neuropathic pain secondary to thermal hyperalgesia, and additionally is effective in treating the hyperalgesia of neuropathic pain due to chronic sciatic nerve constriction injury in rats (Lang 1998). Venlafaxine-induced antinociception is significantly inhibited by naloxone, nor-BNI and naltrindole but not by β-FNA or naloxonazine, implying involvement of κ1- and δ-opioid mechanisms. When adrenergic and serotoninergic antagonists are used, yohimbine but not phentolamine or metergoline, decreased antinociception elicited by venlafaxine, implying a clear α2- and a minor α1-adrenergic mechanism of antinociception. Therefore, the antinociceptive effect of venlafaxine is mainly influenced by the κ- and δ-opioid receptor subtypes combined with the α2-adrenergic receptor. These results suggest a potential use of venlafaxine in the management of some pain syndromes.

B. Dextromethorphan

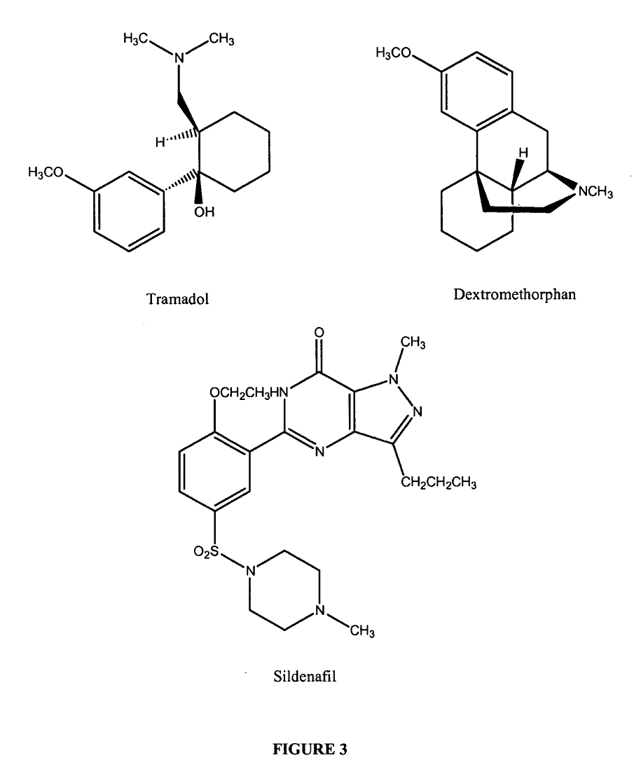

Dextromethorphan (frequently abbreviated as DM) is the common name for(+)-3-methoxy-N-methylmorphinan (FIG. 3). It widely used as a cough syrup, and is described in references such as Rodd 1960 (full citations to articles are provided below) and Goodman and Gilman's Pharmacological Basis of Therapeutics. Briefly, DM is a non-addictive opioid comprising a dextrorotatory enantiomer (mirror image) of the morphinan ring structure which forms the molecular core of most opiates.

DM acts at a class of neuronal receptors known as sigma receptors. These are often referred to as sigma opiate receptors, but there is some question as to whether they are opiate receptors, so many researchers refer to them simply as sigma receptors, or as high-affinity dextromethorphan receptors. They are inhibitory receptors, which means that their activation by DM or other sigma agonists causes the suppression of certain types of nerve signals. Dextromethorphan also acts at another class of receptors known as N-methyl-D-aspartate (NMDA) receptors, which are one type of excitatory amino acid (EAA) receptor. Unlike its agonist activity at sigma receptors, DM acts as an antagonist at NMDA receptors, which means that DM suppresses the transmission of nerve impulses mediated via NMDA receptors. Since NMDA receptors are excitatory receptors, the activity of DM as an NMDA antagonist also leads to the suppression of certain types of nerve signals, which may also be involved in some types of coughing. Due to its activity as an NMDA antagonist, DM and one of its metabolites, dextrorphan, are being actively evaluated as possible treatments for certain types of excitotoxic brain damage caused by ischemia (low blood flow) and hypoxia (inadequate oxygen supply), which are caused by events such as stroke, cardiac arrest, and asphyxia. The anti-excitotoxic activity of dextromethorphan and dextrorphan, and the blockade of NMDA receptors by these drugs, are discussed in items such as Choi (1987), Wong et al, (1988), Steinberg et al, (1988), and U.S. Pat. No. 4,806,543. Dextromethorphan has also been reported to suppress activity at neuronal calcium channels (Carpenter et al, 1988). Dextromethorphan and the receptors it interacts with are further discussed in Tortella et al, (1989), Leander (1989), Koyuncuoglu & Saydam (1990), Ferkany et al, (1988), George et al, (1988), Prince & Feeser (1988), Feeser et al, (1988), Craviso and Musacchio (1983) and Musacchio et al, (1988).

DM disappears fairly rapidly from the bloodstream (see, e.g., Vetticaden et al, (1989) and Ramachander et al, (1977)). DM is converted in the liver to two metabolites called dextrorphan and 3-methoxymorphinan, by an enzymatic process called O-demethylation; in this process, one of the two pendant methyl groups is replaced by hydrogen. If the second methyl group is removed, the resulting metabolite is called 5-hydroxymorphinan. Dextrorphan and 5-hydroxymorphinan are covalently bonded to other compounds in the liver (primarily glucuronic acid or sulfur-containing compounds such as glutathione) to form glucuronide or sulfate conjugates which are eliminated fairly quickly from the body via urine bloodstream. This enzyme is usually referred to as debrisoquin hydroxylase, since it was discovered a number of years ago to carry out a hydroxylation reaction on debrisoquin. It is also referred to in various articles as P450-DB or P450-2D6. It apparently is identical to an enzyme called sparteine monooxygenase, which was shown years ago to metabolize sparteine; it was not until recently that scientists realized that a single isozyme appears to be primarily responsible for oxidizing both debrisoquin and sparteine, as well as dextromethorphan and various other substrates. A number of compounds inhibit the activity of the debrisoquin hydroxylase (sparteine monooxygenase) isozyme; see Inaba et al, (1985).

Debrisoquin hydroxylase belongs to a family of enzymes known as “cytochrome P-450” enzymes, or as “cytochrome oxidase” enzymes. Monooxygenation of chemical materials has been ascribed to cytochromes P450 (P450). These hemoprotein containing monooxygenase enzymes displaying a reduced carbon monoxide absorption spectrum maximum near 450 nm have been shown to catalyze a variety of oxidation reactions including hydroxylation of endogenous and exogenous compounds (Jachau, 1990). An extensive amount of research has been conducted on the mechanism's by which P450's can catalyze oxygen transfer reactions (Testa and Jenner, 1981; Guengerich, 1992; Brosen et al, 1990; Murray et al, 1990; and Porter et al, 1991). The most powerful of these inhibitors is quinidine, a dextrorotatory stereoisomer of quinine; it is normally used to treat cardiac arrhythmias. Inaba et al, (1986) and Nielsen et al, (1990) discuss the ability of quinidine to inhibit the oxidation of sparteine in in vivo animal tests, and Brinn et al, 1986, Brosen et al, 1987, and Broly et al, 1989 discuss the ability of quinidine to inhibit DM metabolism in liver cell preparations. In addition to the inhibition of debrisoquin hydroxylase, which is exceptionally potent and easily demonstrated, other cytochrome P450 isozymes are also likely to be suppressed by quinidine, with varying levels of binding affinity. Accordingly, even though quinidine exerts its most marked effect on debrisoquin hydroxylase, it is likely to suppress a number of other cytochrome P450 enzymes as well, thereby subjecting a patient to a more general loss of normal and desirable liver activity. The primary oxidized metabolic product of dextromethorphan is dextrorphan, which is widely believed among neurologists to be active in exactly the same manner as dextromethorphan; both drugs reportedly are sigma agonists, NMDA antagonists, and calcium channel antagonists.

It has been shown that the administration of a compound which inhibits debrisoquin hydroxylase, in conjunction with DM, causes a major increase in the concentration and stability of DM in the blood of patients, compared to patients who receive only DM; and the administration of a debrisoquin hydroxylase inhibitor in conjunction with DM has a clear and substantial impact on the detectable effects of DM in humans. Eventhough debrisoquin hydroxylase inhibitors may be used to potentiate the activity of dextromethorphan, other agents which inhibit the oxidative activity of cytochrome-P450, such as a naphthyridine, xanthine, phenoxy amino alkane, carbamoyl imidazole, guanidine imidazole, e.g. cimetidine (N-cyano-N′-methyl-N″-[2[[(5-methyl-1H-imidazol-4 yl)methyl]thio]ethyl]guanidine), quinoline, e.g. chloroquine (7-chloro-4-(4-diethylamino-1-methylbutylamino)quinoline) and primaquine (8-(4-amino-1-methylbutylamino)-6-methoxyquinoline), a trifluoromethyl oxime ether, e.g., fluvoxamine, also known as 5-methoxy-1-[4-(trifluoromethyl)-phenyl]-1 pentanone 0-(2-aminoethyl) oxime may be used to potentiate the activity of dextromethorphan.

A non-limiting list of NMDA antagonist drugs which may be utilized in the present invention include dextromethorphan, dextrorphan, ketamine, amantadine, memantine, eliprodil, ifenprodil, phencyclidine, MK-801, dizocilpine, CCPene, flupirtine, or derivatives, salts, metabolites or complexes thereof.

1. Cytochrome P450 Inhibitors

In order to potentiate the effect of dextromethorphan, optionally an effective amount of a cytochrome P450 enzyme inhibitor such as quinidine can be administered to the patient either in a combination dosage unit or in a sequential administration dosage unit. When a cytochrome P450 inhibitor is administered in order to augment the effect of dextromethorphan, the dosage of dextromethorphan can be suitably adjusted to have maximum efficacy with minimum side effects. Oral combination dosage units preferably can contain quinidine in the range of about 50 to not more than 200 milligrams (mg), preferably in the range of about 90 and about 120 mg. Oral combination dosage units preferably can contain quinidine in the range of about 50 to not more than 200 milligrams (mg), preferably in the range of about 90 and about 120 mg.

C. Capsaiciniods

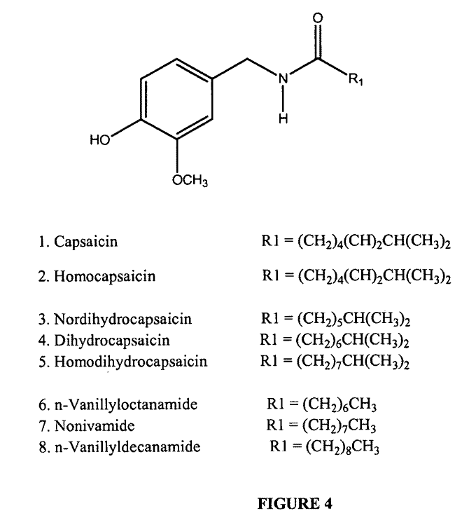

A capsaicinoid may optionally included in a pharmaceutical preparation of the present invention used to treat premature ejaculation. Capsaicin (FIG. 4) is a natural constituent in pungent red chili peppers. Depending on the concentration used and the mode of application, capsaicin can selectively activate, desensitize, or exert a neurotoxic effect on small diameter sensory afferent nerves while leaving larger diameter afferents unaffected (Holzer, 1991; Winter et al, 1995). Sensory neuron activation occurs due to interaction with a ligand-gated nonselective cation channel termed the vanilloid receptor (VR-1) (Caterina et al, 1997), and receptor occupancy triggers Na⁺ and Ca²⁺ ion influx, action potential firing, and the consequent burning sensation associated with spicy food or capsaicin-induced pain. VR1 receptors are present on both C and Aδ fibers, and can be activated by capsaicin and its analogs, heat, acidification, and lipid metabolites (Tominaga et al, 1998; Caterina and Julius, 2001). Desensitization occurs with repeated administration of capsaicin, is a receptor-mediated process, and involves Ca²⁺- and calmodulin-dependent processes and phosphorylation of the cation channel (Winter et al, 1995; Wood and Docherty, 1997).

Capsaicin induces release of substance P and calcitonin gene-related peptide from both peripheral and central terminals of sensory neurons, and desensitization inhibits such release (Holzer, 1991); such inhibition may result from inhibition of voltage-gated Ca²⁺-currents (Docherty et al, 1991; Winter et al, 1995). Desensitization leads to analgesia in rodent paradigms, with specific characteristics of analgesia depending on the dose of capsaicin, route of administration, treatment paradigm (i.e., acute or repeated administration), and age of the animal (Holzer, 1991; Winter et al, 1995). The topical skin application of capsaicin to rodents produces analgesia (Kenins, 1982; Lynn et al, 1992), but variability in outcome can occur due to the concentration, the number of applications, and the different vehicles used that can affect the rate and extent of skin penetration (Carter and Francis, 1991; McMahon et al, 1991).

Viral replication, immune regulation, and induction of various inflammatory and growth-regulatory genes require activation of a nuclear transcription factor (NF)-κ-B. Agents that can block NF-κ-B activation have potential to block downstream responses mediated through this transcription factor. Capsaicin (8-methyl-N-vanillyl-6-nonenamide) has been shown to regulate a wide variety of activities that require NF-κ-B activation (Singh 1996). The pretreatment of human myeloid ML-1a cells with capsaicin blocked TNF-mediated activation of NF-κ-B in a dose- and time-dependent manner. Capsaicin treatment of cells also blocked the degradation of I-κ-B alpha, and thus the nuclear translocation of the p65 subunit of NF-κ-B, which is essential for NF-κ-B activation. TNF-dependent promoter activity of I-κ-B alpha, which contains NF-κ⁻B binding sites, was also inhibited by capsaicin.

The distribution and metabolism of capsaicin and/or dihydrocapsaicin has been studied in rats. Capsaicin is distributed to the brain, spinal cord, liver and blood within 20 minutes of intravenous administration. Oral doses of dihydrocapsaicin in the rat showed metabolic activity associated with its absorption into the portal vein. Capsaicin and dihydrocapsaicin are metabolized in the liver by the mixed-function oxidation system (cytochrome P-450-dependent system). It is assumed that capsaicin is excreted in urine. In rats, most of dihydrocapsaicin is known to be rapidly metabolized and excreted in the urine (Rumsfield and West, 1991).

Oral dosing of rats with capsaicin and dihydrocapsaicin results in an 85% absorption in the jejunum after 3 hours (Rumsfield and West, 1991). With respect to topical applications of capsaicin, it has been estimated that assuming 100% of a topically-applied dose is absorbed into the body, an application of 90 g capsaicin (2 tubes of cream, 0.025% capsaicin) per week would result in a daily exposure of 0.064 mg/kg capsaicin for a 50 kg person. This represents less than 10% of the dietary intake of a typical Indian or That diet (Rumsfield and West, 1991).

Capsaicin is a natural constituent in pungent red chili peppers. Depending on the concentration used and the mode of application, capsaicin can selectively activate, desensitize, or exert a neurotoxic effect on small diameter sensory afferent nerves while leaving larger diameter afferents unaffected (Holzer, 1991; Winter et al, 1995). Sensory neuron activation occurs due to interaction with a ligand-gated nonselective cation channel termed the vanilloid receptor (VR-1) (Caterina et al, 1997), and receptor occupancy triggers Na⁺ and Ca²⁺ ion influx, action potential firing, and the consequent burning sensation associated with spicy food or capsaicin-induced pain. VR1 receptors are present on both C and Aδ fibers, and can be activated by capsaicin and its analogs, heat, acidification, and lipid metabolites (Tominaga et al, 1998; Caterina and Julius, 2001). Desensitization occurs with repeated administration of capsaicin, is a receptor-mediated process, and involves Ca²⁺- and calmodulin-dependent processes and phosphorylation of the cation channel (Winter et al, 1995; Wood and Docherty, 1997).

Capsaicin is believed to cause depolarization of C-fiber polymodal nociceptors (Lynn 1990; Marsh 1987) and release of substance P, which is a neurotransmitter that relays pain signals to the brain. This action may actually increase pain sensation after initial use. However, repeat applications deplete the reserves of substance P at the afferent neurons leading to pain relief (Nolan 1999). Depletion of substance P does not occur immediately. Effective use of the cream (0.075% capsaicin) requires topical application 4 or 5 times daily for a period of at least 4 weeks.

1. Capsaicinoid Esters

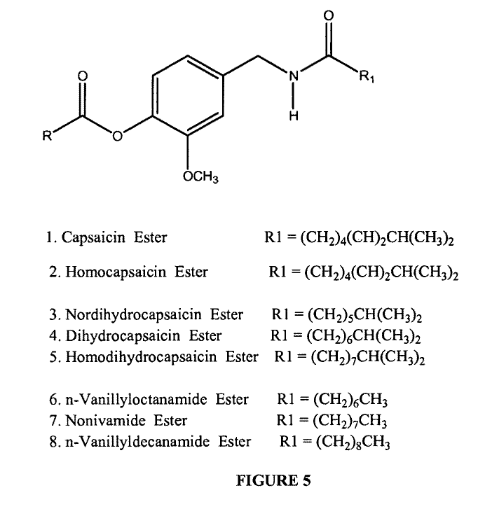

In order to make the capsaicin to have less irritation to the skin and significantly less burning sensation to the stomach, the capsaicin has been esterified at the phenolic position. These esters have the general formula I (see FIG. 5),

R—CO—O₁CAP (I)

wherein CAP refers to a capsaicinoid and O₁CAP refers to an oxygen present in an alcohol group of a corresponding non-esterified capsaicinoid. FIG. 4 and FIG. 5 show examples of non-esterified and esterified capsaicinoids, respectively. Various esterified capsaicinoids are described in US 2008/0020996, which is incorporated by reference in its entirety, and may be used with the present invention. Once administered to a subject, the esterified capsaicinoid may be enzymatically converted to the corresponding capsaicinoid once administered to a subject.

In formula I, R is selected from C₁₋₂₂ alkyl, C₆₋₂₂ aryl, C₁₋₂₂ alkylene, C₁₋₂₂ alkenyl, C₁₋₂₂ alkynyl and/or C₁₋₂₂ arylene. In various embodiments, the alkyl, alkylene, alkenyl, alkynyl and/or arylene may be C₁₋₁₈, C₁₋₁₂, or C₁₋₆. The aryl may be C<2, C<18, C<12, or C=6. The alkyl, aryl and/or alkylene groups may be substituted or unsubstituted, branched or straight chains. In addition, R may contain heteroatoms and may be straight chained or branched.

Examples of suitable straight-chain alkyl groups in formula I include methyl, ethyl, propyl, butyl, hexyl, heptyl, octyl, dodecyl, 1-pentadecyl, 1-heptadecyl and the like groups.

Examples of suitable branched chain alkyl groups in formula I include isopropyl, sec-butyl, t-butyl, 2-methylbutyl, 2-pentyl, 3-pentyl and the like groups.

Examples of suitable cyclic alkyl groups in formula I include cyclopropyl, cyclobutyl, cyclopentyl and cyclohexyl groups.

Examples of suitable “alkenyl” groups in formula I include vinyl (ethenyl), 1-propenyl, i-butenyl, pentenyl, hexenyl, n-decenyl and c-pentenyl and the like.

The groups may be substituted, generally with 1 or 2 substituents, wherein the substituents are independently selected from halo, hydroxy, alkoxy, amino, mono- and dialkylamino, nitro, carboxyl, alkoxycarbonyl, and cyano groups.

By the expression “phenalkyl groups wherein the alkyl moiety contains 1 to 3 or more carbon atoms” is meant benzyl, phenethyl and phenylpropyl groups wherein the phenyl moiety may be substituted. When substituted, the phenyl moiety of the phenalkyl group may contain independently from 1 to 3 or more alkyl, hydroxy, alkoxy, halo, amino, mono- and dialkylamino, nitro, carboxyl, alkoxycarbonyl and cyano groups.

Examples of suitable “heteroaryl” in formula I are pyridinyl, thienyl or imidazolyl.

As noted herein, the expression “halo” is meant in the conventional sense to include F, Cl, Br, and I.

Among the compounds represented by the general Formula I, preferred compounds are such in which R is one of the following groups: methyl, ethyl, propyl, butyl, pentyl, hexyl, 1-pentadecyl, 1-heptadecyl, isobutyl, methoxyethyl, ethoxyethyl, benzyl and nicotinyl.

The compounds esters of capsaicin can be prepared by any method known to those of ordinary skill in the art. For example, the compounds of the present invention are esters of capsaicin which are the constituents of capsicum. Various methods have been described in the literature pertaining to the synthesis of a number of esters of carboxylic acids and phenols (March's Advanced Organic Chemistry: Reactions, Mechanisms, and Structure, 5th Edition, by Michael B. Smith and Jerry March, John Wiley and Sons, Inc, 2001).

2. Chemical Definitions

For the groups below, the following parenthetical subscripts further define the groups as follows: “(Cn)” defines the exact number (n) of carbon atoms in the group; “(Cn)” defines the maximum number (n) of carbon atoms that can be in the group; (Cn−n′) defines both the minimum (n) and maximum number (n′) of carbon atoms in the group. For example, “alkoxy(c<10)” designates those alkoxy groups having from 1 to 10 carbon atoms (e.g., 1, 2, 3, 4, 5, 6, 7, 8, 9, or 10, or any range derivable therein (e.g., 3-10 carbon atoms)). Similarly, “alkyl_{(C2-10)}” designates those alkyl groups having from 2 to 10 carbon atoms (e.g., 2, 3, 4, 5, 6, 7, 8, 9, or 10, or any range derivable therein (e.g., 3-10 carbon atoms)).

The term “alkyl” when used without the “substituted” modifier refers to a non-aromatic monovalent group, having a saturated carbon atom as the point of attachment, a linear or branched, cyclo, cyclic or acyclic structure, no carbon-carbon double or triple bonds, and no atoms other than carbon and hydrogen. The groups, CH₃ (Me), CH₂CH₃ (Et), CH₂CH₂CH₃ (n-Pr), CH(CH₃)₂ (iso-Pr), —CH(CH₂)₂ (cyclopropyl), —CH₂CH₂CH₂CH₃ (n-Bu), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), cyclobutyl, cyclopentyl, cyclohexyl, and cyclohexylmethyl are non-limiting examples of alkyl groups. The term “substituted alkyl” refers to a non-aromatic monovalent group, having a saturated carbon atom as the point of attachment, a linear or branched, cyclo, cyclic or acyclic structure, no carbon-carbon double or triple bonds, and at least one atom independently selected from the group consisting of N, O, F, Cl, Br, I, Si, P, and S. The following groups are non-limiting examples of substituted alkyl groups: —CH₂OH, —CH₂Br, —CH₂SH, —CF₃, —CH₂CN, —CH₂C(O)H, —CH₂C(O)OH, —CH₂C(O)OCH₃, —CH₂C(O)NH₂, —CH₂C(O)NHCH₃, —CH₂C(O)CH₃, —CH₂OCH₃, —CH₂OCH₂CF₃, —CH₂OC(O)CH₃, —CH₂NH₂, —CH₂NHCH₃, —CH₂N(CH₃)₂, —CH₂CH₂Cl, —CH₂CH₂OH, —CH₂CF₃, —CH₂CH₂OC(O)CH₃, —CH₂CH₂NHCO₂C(CH₃)₃, and —CH₂Si(CH₃)₃.

The term “alkenyl” when used without the “substituted” modifier refers to a monovalent group, having a nonaromatic carbon atom as the point of attachment, a linear or branched, cyclo, cyclic or acyclic structure, at least one nonaromatic carbon-carbon double bond, no carbon-carbon triple bonds, and no atoms other than carbon and hydrogen. Non-limiting examples of alkenyl groups include: —CH═CH₂ (vinyl), —CH═CHCH₃, —CH═CHCH₂CH₃, —CH₂CH═CH₂ (allyl), —CH₂CH═CHCH₃, and —CH═CH—C₆H₅. The term “substituted alkenyl” refers to a monovalent group, having a nonaromatic carbon atom as the point of attachment, at least one nonaromatic carbon-carbon double bond, no carbon-carbon triple bonds, a linear or branched, cyclo, cyclic or acyclic structure, and at least one atom independently selected from the group consisting of N, O, F, Cl, Br, I, Si, P, and S. The groups, —CH═—CHF, —CH═CHCl and —CH═CHBr, are non-limiting examples of substituted alkenyl groups.

The term “alkynyl” when used without the “substituted” modifier refers to a monovalent group, having a nonaromatic carbon atom as the point of attachment, a linear or branched, cyclo, cyclic or acyclic structure, at least one carbon-carbon triple bond, and no atoms other than carbon and hydrogen. The groups, —C≡CH, —C≡CCH₃, —C≡CC₆H₅ and —CH₂C≡CCH₃, are non-limiting examples of alkynyl groups. The term “substituted alkynyl” refers to a monovalent group, having a nonaromatic carbon atom as the point of attachment and at least one carbon-carbon triple bond, a linear or branched, cyclo, cyclic or acyclic structure, and at least one atom independently selected from the group consisting of N, O, F, Cl, Br, I, Si, P, and S. The group, —C≡CSi(CH₃)₃, is a non-limiting example of a substituted alkynyl group.

The term “aryl” when used without the “substituted” modifier refers to a monovalent group, having a aromatic carbon atom as the point of attachment, said carbon atom forming part of a six-membered aromatic ring structure wherein the ring atoms are all carbon, and wherein the monovalent group consists of no atoms other than carbon and hydrogen. Non-limiting examples of aryl groups include phenyl (Ph), methylphenyl, (dimethyl)phenyl, —C₆H₄CH₂CH₃ (ethylphenyl), —C₆H₄CH₂CH₂CH₃ (propylphenyl), —C₆H₄CH(CH₃)₂, —C₆H₄CH(CH₂)₂, —C₆H₃(CH₃)CH₂CH₃ (methylethylphenyl), —C₆H₄CH═CH₂ (vinylphenyl), —C₆H₄CH═CHCH₃, C₆H₄C≡CH, —C₆H₄C≡CCH₃, naphthyl, and the monovalent group derived from biphenyl. The term “substituted aryl” refers to a monovalent group, having a aromatic carbon atom as the point of attachment, said carbon atom forming part of a six-membered aromatic ring structure wherein the ring atoms are all carbon, and wherein the monovalent group further has at least one atom independently selected from the group consisting of N, O, F, Cl, Br, I, Si, P, and S, Non-limiting examples of substituted aryl groups include the groups: —C₆H₄F, —C₆H₄Cl, —C₆H₄Br, —C₆H₄I, —C₆H₄OH, —C₆H₄OCH₃, —C₆H₄OCH₂CH₃, —C₆H₄OC(O)CH₃, —C₆H₄NH₂, —C₆H₄NHCH₃, —C₆H₄N(CH₃)₂, —C₆H₄CH₂OH, —C₆H₄CH₂OC(O)CH₃, —C₆H₄CH₂NH₂, —C₆H₄CF₃, —C₆H₄CN, —C₆H₄CHO, —C₆H₄CHO, —C₆H₄C(O)CH₃, —C₆H₄C(O)C₆H₅, —C₆H₄CO₂H, —C₆H₄CO₂CH₃, —C₆H₄CONH₂, —C₆H₄CONHCH₃, and —C₆H₄CON(CH₃)₂.

An “isomer” of a first compound is a separate compound in which each molecule contains the same constituent atoms as the first compound, but where the configuration of those atoms in three dimensions differs.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylicacids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, Selection and Use (P. H. Stahl & C. G. Wermuth eds., Verlag Helvetica Chimica Acta, 2002), which is incorporated herein by reference.

“Prevention” or “preventing” when used in reference to a disease includes: (1) inhibiting the onset of the disease in a subject or patient which may be predisposed to the disease but does not yet experience or display the pathology or symptomatology of the disease, (2) slowing the onset of the pathology or symptomatology of the disease in a subject of patient which may be predisposed to the disease but does not yet experience or display the pathology or symptomatology of the disease.

“Prodrug” means a compound that is convertible in vivo metabolically into an inhibitor according to the present invention. In addition to esterified capsaicinoids, it is envisioned that other capsaicinoid prodrugs may be used with the present invention. The prodrug itself may or may not also have activity with respect to a given target protein or therapeutic effect. For example, a compound comprising a hydroxy group may be administered as an ester that is converted by hydrolysis in vivo to the hydroxy compound. As described herein, amyris alcohol prodrugs such as esterified amyris alcohols are provided for the treatment of diseases including herpes virus infection. Suitable esters that may be converted in vivo into hydroxy compounds include acetates, citrates, lactates, phosphates, tartrates, malonates, oxalates, salicylates, propionates, succinates, fumarates, maleates, methyl ene-bis-b-hydroxynaphthoates, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Amyris alcohols may be esterified using any of these approaches, and it is envisioned that these esterified amyris alcohols may be used with the present invention (e.g., to treat a herpesvirus infection, etc.) Similarly, a compound comprising an amine group may be administered as an amide that is converted by hydrolysis in vivo to the amine compound.

The term “saturated” when referring to a atom means that the atom is connected to other atoms only by means of single bonds.

The terms “subject” and “patient” includes humans, primates and other mammals.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers.

“Therapeutically effective amount” means that amount which, when administered to an animal for treating a disease, is sufficient to effect such treatment for the disease.

“Treatment” or “treating” includes: (1) inhibiting a disease in an subject or patient that is experiencing or displaying the pathology or symptomatology of the disease (i.e., arresting further development of the pathology and/or symptomatology), and (2) ameliorating the disease in a subject or patient that is experiencing or displaying the pathology or symptomatology of the diseased (i.e., reversing the pathology and/or symptomatology).

3. Synthesis of a Capsaicinoid Ester

One method that has been utilized for efficient preparation of an esterified capsaicinoid of the present invention is through dissolution of the compound in methylene dichloride. Since capsaicin USP27 contains >95% of capsaicins, to this solution slightly in excess of 1.1 mole equivalent of anhydrous triethylamine is added with stirring at room temperature and the mixture is kept around room temperature. To this solution slightly in excess of 1 mole equivalent of an acid chloride is added with stirring while keeping the temperature around 20-25° C. and the solution was refluxed for 5-6 hours and stirred for 12-16 hours at room temperature. The organic phase was washed 3-4 times with water and then 2 times with 7% sodium carbonate solution in a separating funnel to remove any acid present in the organic solution. The organic phase was then washed 2-3 times with dilute hydrochloric acid solution in a separating funnel to remove any amine present in the organic solution. The organic phase was then washed with equal amount of water three to four times until the pH of the aqueous phase is around 6-7. The organic phase was dried with anhydrous sodium sulfate overnight and the methylene dichloride was removed in a rotary evaporator under vacuum. The resultant oily or waxy material is called the ester capsaicin as all of the phenols present in capsaicin is converted into the corresponding ester.

The compounds of Formula I are esters of capsaicin present in capsicum. For oral administration, the preferred ester is the palmitate esters of capsaicin. These esters have less irritation and burning sensation to the stomach and are used for to control neuronal sensation through its binding to the VR1 receptors and the depletion of substance P.

A non-limiting list of capsaicin which may be used in the present invention include capsaicin, homocapsaicin, nordihydrocapsaicin, dihydrocapsaicin, homodihydrocapsaicin or any compounded mixture thereof.

A non-limiting list of an ester of capsaicin which may be used in the present invention includes capsaicin palmitate.

D. Dosages

Oral combination dosage units preferably contain dextromethorphan in the range of about 30 to not more than 200 milligrams (mg), preferably in the range of about 60 and about 120 mg and tramadol in the range of about 30 to about 500 mg, preferably in the range of about 30 to about 200 mg, so long as the combined dose received by the patient is accompanied by minimal or substantially no undesirable side effects. Capsaicin or capsaicin palmitate can be in the composition at a dosage of about 2 to not more than 50 mg, preferably in the range of about 5 mg and about 20 mg.

In certain embodiments, the following oral combination dosages are used: about 100 mg dextromethorphan and not more than 100 mg tramadol, more preferably about 45 mg dextromethorphan and about 35 mg tramadol and about 3 mg of capsaicin or 6 mg of capsaicin palmitate.

Alternatively, the dextromethorphan and tramadol may be formulated separately in the foregoing compositions as the sole active ingredient for practicing sequential administration of each respective drug.

Alternatively, the dextromethorphan, capsaicin or capsaicin palmitate and tramadol may be formulated separately in the foregoing compositions as the sole active ingredient for practicing sequential administration of each respective drug.

For sequential administration therapy, tramadol, capsaicin or capsaicin palmitate and dextromethorphan are administered in a separate dosage. For sequential administration of tramadol, the dosage unit preferably contains tramadol in a range of about 10 to about 500 mg, more preferably in the range of about 20 mg to about 200 mg, for administration of capsaicin palmitate, the dosage unit preferably contains in a range of about 5 to about 50 mg, more preferably in the range of about 5 mg to about 20 mg and for administration of dextromethorphan the dosage unit preferably contains dextromethorphan in a range of about 30 to not more than 120 mg, more preferably in the range of about 50 to about 90 mg so long as the total combined dose received by the patient is accompanied by minimal or substantially no undesirable side effects.

A particularly preferred sequential administration dosage unit contains tramadol in the range of about 30 to about 100 mg and dextromethorphan in the range of about 30 to about 135 mg. Preferably, each drug is administered orally. Alternatively, each drug can be administered by different oral routes; i.e., one can be ingested and the other administered sublingually or by buccal patch.

For effective sequential administration of tramadol, capsaicin palmitate and dextromethorphan, the release of each drug is preferably staggered to maximize the beneficial delayed ejaculation by dextromethorphan. For example, tramadol and capsaicin palmitate can be administered simultaneously and dextromethorphan can be administered within 30-90 minutes.

For effective sequential administration of tramadol, capsaicin palmitate and dextromethorphan, the release of each drug may be staggered to maximize the beneficial delayed ejaculation by dextromethorphan.

Dosage levels of the NMDA antagonist on the order of from about 0.3 mg to about 3 mg per kilogram of body weight per day are examples of therapeutically effective doses when administered in combination with tramadol or its analog. Alternatively, about 10 mg to about 200 mg per patient per day of a NMDA antagonist may administered in combination with tramadol or its analog.

The amount of NMDA antagonist that may be combined with the carrier materials to produce a single dosage form having NMDA antagonist and tramadol or its analog in combination will vary depending upon the patient and the particular mode of administration. For example, a formulation intended for the oral administration of humans may contain from 10 mg to 300 mg of NMDA antagonist compounded with an appropriate and convenient amount of carrier material that may vary from about 5 to about 95 percent of the total composition. Unit dosages may generally contain between from about 10 mg to about 100 mg of a NMDA antagonist.

Tramadol or its analog can be provided in a oral dosage form with as the therapeutically active agent in an amount from about 25 mg to about 400 mg tramadol hydrochloride. Alternatively, the dosage form may contain molar equivalent amounts of other tramadol salts or of the tramadol base. The dosage form may contain a mixture of tramadol and its analog to provide a substantially equivalent therapeutic effect.

The amount of capsaicin palmitate in the composition is preferably an amount sufficient to further enhance therapeutic effect or to hasten the onset of therapeutic effects. In humans, this amount may be from about 1 to about 100 mg (preferably 5 to 30 mg), which can be sufficient to both hasten onset and enhance analgesia. The daily dosage of capsaicin palmitate again will generally not exceed 100 mg. Of course, greater amounts can be used if tolerated by the patient.

In certain embodiments, sequential administration is accomplished by administering to a subject: tramadol in the range of about 30 to about 100 mg, capsaicin palmitate in the range of 5 to 50 mg and dextromethorphan in the range of about 30 to about 135 mg. Preferably, each drug is administered orally. Alternatively, each drug can be administered by different oral routes; i.e., one can be ingested and the other administered sublingually or by buccal patch.

### II. TREATMENTS FOR PREMATURE EJACULATION IN CLASS II MALES

In order to carry out the method of the invention to treat premature ejaculation in CLASS II males who have erection as well as ejaculation problems, the above pharmaceutical compositions may be modified to include administration of an active agent such as a phosphodiesterase type 5 (PDE5) inhibitor to promote an erection in a male. Thus, cyclic-GMP-specific phosphodiesterase type 5 (PDE5) inhibitors such as sildenafil, capsaicin palmitate, an anti-excitotoxic agent such as dextromethorphan, and tramadol or a derivative or analog of tramadol may be administered to a CLASS II male to treat premature ejaculation. The active agents may be administered orally, parenterally, buccally, rectally, or locally by intracavernosal injection or by delivery to the urethra.

A. Phosphodiesterase Type 5 (PDE5) Inhibitors

Sildenafil is designated chemically as 1-[[3-(6,7-dihydro-1-methyl-7-oxo-3-propyl-1H-pyrazolo[4,3-d]pyrimidin-5-yl)-4-ethoxyphenyl]sulfonyl]-4-methyl piperazine and has the following structural formula: FIG. 3.

Sildenafil citrate is presently the active ingredient of a commercial medication for impotence sold under the designation Viagra™ (Pfizer Labs, N.Y.) formulated in tablets equivalent to 25 mg, 50 mg and 100 mg sildenafil for oral administration. According to the manufacturer, in addition to the active ingredient, sildenafil citrate, each tablet contains the following inactive ingredients: microcrystalline cellulose, anhydrous dibasic calcium phosphate, croscarmellose sodium, magnesium stearate, hydroxypropyl methylcellulose, titanium dioxide, lactose, triacetin, and FD&C Blue #2 aluminum lake.

It is known from in vitro studies that sildenafil is approximately 4,000 fold more selective for inhibiting phosphodiesterase type 5 (PDE5) than on other known phosphodiesterases, such as PDE3, which is involved in control of cardiac contractility. Sildenafil is reportedly only about 10-fold as potent for PDE5 compared to PDE6, an enzyme found in the retina and it is this lower selectivity which is thought to be the basis for abnormalities related to color vision observed with higher doses or plasma levels.

Sildenafil, administered as the commercially available Viagra™ formulation, is reported to be rapidly absorbed after oral administration, with absolute bioavailability of about 40%. Its pharmacokinetics are dose-proportional over the recommended dose range. Based on the Viagra™ manufacturer's product literature, maximum observed plasma concentrations are reached within 30 to 120 minutes (median 60 minutes) of oral dosing in the fasted state. When the Viagra™ formulation is taken with a high fat meal, the rate of absorption is reduced, with a mean delay in Tmax of 60 minutes and mean reduction in Cmax of 29%. The mean steady state volume of distribution (Vss) for sildenafil is reportedly 105 L, indicating distribution into the tissues. Based upon reported measurements of sildenafil in the semen of healthy volunteers 90 minutes after dosing, less than 0.001% of the administered dose appeared in the semen of the patients.

Hull et al (1994) observed that nitric oxide (NO) may inhibit seminal emission in male rats, probably by decreasing sympathetic nervous system activity. Kriegsfeld et al (1999) noted that mice lacking endothelial NO synthase (eNOS) showed a higher incidence of premature ejaculation. In addition, Heuer et al. (2002) observed in vitro that the NO-cGMP cascade in part regulates human seminal vesicle contractility. Furthermore, it has been suggested that nitric oxide activity in the medial preoptic area tonically inhibits ejaculation by decreasing sympathetic tone (Pfaus 1999). These are rationales for using NO donating drugs as pharmacotherapy for PE. Sildenafil is a selective inhibitor of cyclic guanosine monophosphate (cGMP) specific phosphodiesterase type 5, which has been approved as a first line oral therapy for erectile dysfunction (Goldstein 1998; McMahon 2000). It thus enhances the relaxant effect of nitric oxide released in response to sexual stimulation by increasing cGMP concentrations in the corporal smooth muscle (Padma-Nathan 1999). In a study sildenafil administered as needed as a single treatment for PE, increased ejaculation time more than paroxetine (Abdel-Hamid 2001). In contrast, clomipramine, sertraline and paroxetine appear to be comparable in terms of efficacy. A number of studies suggest that adding a PDE5 inhibitor such as sildenafil to an SSRI such as paroxetine is better for PE than either drug alone (Abdel-Hamid 2004; Salonia 2002). Abdel-Hamid attributed the positive result associated with sildenafil use to following possible mechanisms. The first may be possible reduction in performance anxiety and the second is that sildenafil may maintain erection and increase the erection time, and ejaculation latency time was reported to be dependent on erection time

Surprisingly, a therapeutically effective dosage combination of dextromethorphan, tramadol, sildenafil and optionally capsaicin palmitate of this invention maximizes the beneficial erectogenic efficacy of sildenafil by delaying the premature ejaculation.

B. Dosages

Oral combination dosage units preferably contain dextromethorphan in the range of about 10 to not more than 300 milligrams (mg), preferably in the range of about 30 and about 200 mg, tramadol in the range of about 10 to not more than 200 milligrams (mg), preferably in the range of about 30 and about 150 mg, capsaicin palmitate in the range of about 5 to not more than 50 milligrams (mg), preferably in the range of about 5 and about 20 mg and of sildenafil in the range of about 10 to about 200 mg, preferably in the range of about 15 to about 100 mg. Generally, the combined dosages received by the patient will be chosen to minimize or essentially eliminate any undesirable side effects.

In certain embodiments, the following oral combination dosages may be used: about 150 mg dextromethorphan, not more than 200 mg of tramadol, more preferably about 100 mg of tramadol and not more than 150 mg sildenafil, more preferably about 135 mg dextromethorphan, about 100 mg of tramadol, about 20 mg of capsaicin palmitate and about 100 mg sildenafil.

Alternatively, the dextromethorphan, tramadol, capsaicin palmiate and sildenafil may be formulated separately in the foregoing compositions as the sole active ingredient for practicing sequential administration of each respective drug.

For sequential administration therapy, sildenafil, tramadol and dextromethorphan each is administered in a separate dosage. For sequential administration of sildenafil, the dosage unit preferably contains sildenafil in a range of about 10 to about 300 mg, more preferably in the range of about 25 to about 200 mg, for administration of tramadol, the dosage unit preferably contains tramadol in a range of about 20 to not more than 400 mg, more preferably in the range of about 30 to about 200 mg and for administration of dextromethorphan the dosage unit preferably contains dextromethorphan in a range of about 30 to not more than 500 mg, more preferably in the range of about 60 to about 300 mg. Dosages may be chosen by a practitioner to minimize or essentially eliminate any undesirable side effects.

A particularly preferred sequential administration dosage unit of sildenafil contains sildenafil in the range of about 50 to about 150 mg, of tramadol contains tramadol in the range of about 50 to about 200 mg and of dextromethorphan contains dextromethorphan in the range of about 45 to about 200 mg. Preferably, each drug is administered orally. Alternatively, each drug can be administered by different oral routes; i.e., one can be ingested and the other administered sublingually or by buccal patch.

For effective sequential administration of sildenafil, tramadol and dextromethorphan, the release of each drug is preferably staggered to maximize the beneficial prolongation of erection by dextromethorphan and tramadol and maintenance of erection by sildenafil upon sexual stimulation.

To augment the beneficial effect of dextromethorphan, tramadol and sildenafil therapy, lesser amounts of erectogenic agents can be included. The term “erectogenic agents” as used herein refers to adrenal steroids, such as testosterone, dehydroepiandrosterone (DHEA) and the like. Preferably, the erectogenic agents are added in an amount in the range of about 5 to about 10 percent by weight, more preferably in the range of about 6 to about 8 percent by weight of the weight of sildenafil administered.

For example, tramadol and dextromethorphan can be administered simultaneously and sildenafil can be administered after 30 to 120 minutes of administering tramadol and dextromethorphan.

### III. PHARMACEUTICAL PREPARATIONS

Pharmaceutical compositions of the present invention comprise an effective amount of the above compounds for the treatment of premature ejaculation or additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one or more of the above compounds for the treatment of premature ejaculation or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The above compounds for the treatment of premature ejaculation may comprise different types of carriers depending on whether it is to be administered in solid, liquid or aerosol form, and whether it need to be sterile for such routes of administration as injection. The present invention can be administered intradermally, transdermally, intravenously, intranasally, intrarectally, topically, intramuscularly, subcutaneously, mucosally, orally, locally, inhalation (e.g., aerosol inhalation), injection, or by other method or any combination of the forgoing as would be known to one of ordinary skill in the art (see, for example, Remington's Pharmaceutical Sciences, 18th Ed. Mack Printing Company, 1990, incorporated herein by reference).

Depending on the intended mode of administration, the pharmaceutical compositions may be in the form of solid, semi-solid or liquid dosage forms, such as, for example, tablets, suppositories, pills, capsules, powders, liquids, suspensions, creams, ointments, lotions or the like, preferably in unit dosage form suitable for single administration of a precise dosage. The compositions will include an effective amount of the selected drugs in combination with a pharmaceutically acceptable carrier and, in addition, may include other pharmaceutical agents, adjuvants, diluents, buffers, etc. The compounds may thus be administered orally, parenterally, transdermally, rectally, nasally, buccally, topically or via an implanted reservoir in dosage formulations containing conventional non-toxic pharmaceutically acceptable carriers, adjuvants and vehicles. The term “parenteral” as used herein is intended to include subcutaneous, intravenous, and intramuscular injection. The amount of active compound administered will, of course, be dependent on the subject being treated, the subject's weight, the manner of administration and the judgment of the prescribing physician.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, etc., an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered may also contain minor amounts of nontoxic auxiliary substances such as wetting or emulsifying agents, pH buffering agents and the like, for example, sodium acetate, sorbitan mono-laurate, triethanolamine sodium acetate, triethanolamine oleate, etc. Actual methods of preparing such dosage forms are known, or will be apparent, to those skilled in this art; for example, see Remington's Pharmaceutical Sciences, referenced above. For oral administration, the composition will generally take the form of a tablet or capsule, or may be an aqueous or nonaqueous solution, suspension or syrup. Tablets and capsules are preferred oral administration forms. Tablets and capsules for oral use will generally include one or more commonly used carriers such as lactose and corn starch. Lubricating agents, such as magnesium stearate, are also typically added. When liquid suspensions are used, the active agent may be combined with emulsifying and suspending agents. If desired, flavoring, coloring and/or sweetening agents may be added as well. Other optional components for incorporation into an oral formulation herein include, but are not limited to, preservatives, suspending agents, thickening agents, and the like.

Parenteral administration, if used, is generally characterized by injection. Injectable formulations can be prepared in conventional forms, either as liquid solutions or suspensions, solid forms suitable for solubilization or suspension in liquid prior to injection, or as emulsions. Preferably, sterile injectable suspensions are formulated according to techniques known in the art using suitable carriers, dispersing or wetting agents and suspending agents. The sterile injectable formulation may also be a sterile injectable solution or a suspension in a nontoxic parenterally acceptable diluent or solvent. Among the acceptable vehicles and solvents that may be employed are water, Ringer's solution and isotonic sodium chloride solution. In addition, sterile, fixed oils, fatty esters or polyols are conventionally employed as solvents or suspending media. A more recently revised approach for parenteral administration involves use of a slow release or sustained release system, such that a constant level of dosage is maintained. See, e.g., U.S. Pat. No. 3,710,795.

The active agent can be administered in a pharmaceutical formulation suitable for transurethral drug delivery. The formulation contains one or more selected carriers or excipients, such as water, silicone, waxes, petroleum jelly, polyethylene glycol (“PEG”), propylene glycol (“PG”), liposomes, sugars such as mannitol and lactose, and/or a variety of other materials, with polyethylene glycol and derivatives thereof particularly preferred. Depending on the drug administered, it may be desirable to incorporate a transurethral permeation enhancer in the urethral dosage form. Examples of suitable transurethral permeation enhancers include dimethylsulfoxide (“DMSO”), dimethyl formamide (“DMF”), N,N-dimethylacetamide (“DMA”), decylmethylsulfoxide (“C10 MSO”), polyethylene glycol monolaurate (“PEGML”), glycerol monolaurate, lecithin, the 1-substituted azacycloheptan-2-ones, particularly 1-n-dodecylcyclazacycloheptan-2-one (available under the trademark Azone® from Nelson Research & Development Co. Irvine, Calif.), SEPA® (available from Macrochem Co., Lexington, Mass.), alcohols (e.g., ethanol), detergents (such as Tergitol®, Nonoxynol-9® and TWEEN-80®) and the like.

A. Cyclodextrins

If desired, to facilitate absorption and thus bioavailability, absorption enhancing agents, such as cyclodextrins, particularly (3-cyclodextrin, or a derivative thereof, such as hydroxypropyl-β-cyclodextrin (HPBCD) and the like may be included. Cyclodextrins are a group of cyclic, nonreducing oligosaccharides built up from six, seven or eight glucopyranose rings, respectively known as alpha, beta and gamma cyclodextrins. The cyclodextrins are a class of cavity-containing cyclic compounds possessing the property of forming a molecular inclusion complexes, which anchor or entrap another chemical compounds without the formation of covalent bonds. HPBCD is a cyclic polymer having a doughnut-shaped molecular structure including an inner cavity.

Hydroxypropyl-β-cyclodextrins are commercially available compounds that are derived from (3-cyclodextrins by condensation with a propylene oxide to provide the corresponding hydroxypropyl derivatives having a degree of substitution (D.S.) of up to about 15 or higher. For the purposes of the present invention a D.S. value of about 5 to 7 is preferred.

The preparation of such suitable hydroxypropyl-β-cyclodextrin (HPBCD) is described, inter alias, in the International Journal of Pharmaceutics, 29, 73-82 (1986) and in the Journal of Pharmaceutical Sciences, 75 (6), 571-572 (1986). Also known and suitable for the present invention are the hydroxypropyl-β-cyclodextrins that are polyethers of cyclodextrins and are obtained by the condensation of an excess of hydroxypropylene oxide with β-cyclodextrin as described in U.S. Pat. No. 3,459,731. or Gramera et al. HPBCD may be used in certain embodiments as a cyclodextrin constituent, although it is envisioned that other cyclodextrins may be used to achieve a similar effect. The weight percent of the HPBCD in the composition is preferably in the range of about 1 to about 10 weight percent of the total composition.

Particularly in the case of sildenafil, it has been found that HPBCD can enhance bioavailability. Thus, the desired therapeutic effect can be achieved with a relatively lower dose of sildenafil, thereby minimizing the likelihood of adverse affects.

B. Prodrugs

The active agents may be administered in the form of pharmaceutically acceptable salts, esters, amides or prodrugs or combinations thereof. However, conversion of inactive ester, amide or prodrug forms to an active form may occur prior to or upon reaching the target tissue or cell. Salts, esters, amides and prodrugs of the active agents may be prepared using standard procedures known to those skilled in the art of synthetic organic chemistry and described, for example, by J. March, Advanced Organic Chemistry: Reactions, Mechanisms and Structure, 4th Ed. (New York: Wiley-Interscience, 1992). For example, acid addition salts are prepared from the free base (typically wherein the neutral form of the drug has a neutral —NH2 group) using conventional means, involving reaction with a suitable acid. Generally, the base form of the drug is dissolved in a polar organic solvent such as methanol or ethanol and the acid is added thereto. The resulting salt either precipitates or may be brought out of solution by addition of a less polar solvent. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, ptoluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Conversely, preparation of basic salts of acid moieties which may be present on a drug are prepared in a similar manner using a pharmaceutically acceptable base such as sodium hydroxide, potassium hydroxide, ammonium hydroxide, calcium hydroxide, trimethylamine, or the like. Preparation of esters involves functionalization of hydroxyl and/or carboxyl groups which may be present within the molecular structure of the drug. The esters are typically acyl-substituted derivatives of free alcohol groups, i.e., moieties which are derived from carboxylic acids of the formula RCOOH where R is alkyl, and preferably is lower alkyl. Esters can be reconverted to the free acids, if desired, by using conventional hydrogenolysis or hydrolysis procedures. Preparation of amides and prodrugs can be carried out in an analogous manner. Other derivatives and analogs of the active agents may be prepared using standard techniques known to those skilled in the art of synthetic organic chemistry, or may be deduced by reference to the pertinent literature. In addition, chiral active agents may be in enantiomerically pure form, or they may be administered as an enantiomeric mixture.

### IV. EXAMPLES

It is to be understood that while the invention has been described in conjunction with the preferred specific embodiments thereof, that the foregoing description as well as the examples which follow are intended to illustrate and not limit the scope of the invention. Other aspects, advantages and modifications within the scope of the invention will be apparent to those skilled in the art to which the invention pertains.

### Example 1

### Preparation of Palmitate Ester of Capsaicin USP27

### Formula I, R═CH₃—(CH₂)₁₄

A mixture of 30.5 gm (˜0.1M) of capsaicin USP27 (HUBEI XIANGXI CHEMICAL INDUSTRY CO., LTD, China), 16.7 ml (0.12M) of anhydrous triethylamine (Spectrum Chemicals), 220 mg of 4-(dimethylamino)pyridine and 200 ml of anhydrous dichloromethane was placed into a 1000 ml 2-neck round bottomed flask. The content was covered with aluminum foil to protect it from light exposure. The flask was fitted with a condenser fitted with a moisture trap on the top and a dropwise addition funnel. The flask was kept at room temperature and 25.4 ml (0.095M) of palmitoyl chloride was added from the funnel into the mixture slowly with stirring. After the addition, the mixture was refluxed for 3-6 hours and stirred for 10-15 hours at room temperature. The mixture was transferred into a separating funnel and washed successively with 2×500 ml of water, 2×500 ml of dilute hydrochloric acid, 2×500 ml of 10% sodium bicarbonate solution and 3×500 ml of type I water. The organic layer was separated, dried with anhydrous magnesium sulfate and the dichloromethane was removed under vacuum to produce a light yellow solid (95% of theoretical). The light yellow solid, as obtained above, was re-crystallized from ethanol. In a 2-liter flask, the solid was dissolved in 1 liter of hot ethanol and filtered through a filter paper. The filtrate was then cooled in the refrigerator to get pale yellow crystals.

### Example 2

### Capsule Formulation

The following ingredients in each one of the capsule formulations were weighed accurately, ground using a pestle and mortar to fine and homogeneous powders. These powders were sieved through 100 mesh and filled into hard gelatin capsules. The composition of each capsule formulation is listed below.

CAPSULE FORMULATION 1

| | In each | In 100 |
|---|---|---|
| Tramadol Hydrochloride | 39.8 mg | 3.98 g |
| Dextromethorphan Hydrochloride | 51.0 mg | 5.10 g |
| Ascorbyl Palmitate | 20.0 mg | 2.00 g |
| Microcrystalline Cellulose | 96.2 mg | 9.62 g |
| Sodium Lauryl Sulfate | 1.5 mg | 0.15 g |
| Silicon dioxide | 1.5 mg | 0.15 g |
| Total Solid | 210 mg | 21.0 g |

CAPSULE FORMULATION 2

| | In each | In 100 |
|---|---|---|
| Tramadol Hydrochloride | 56.9 mg | 5.69 g |
| Dextromethorphan Hydrochloride | 51.0 mg | 5.10 g |
| Ascorbyl Palmitate | 25.0 mg | 2.50 g |
| Starch | 49.1 mg | 4.91 g |
| Lactose | 25.0 mg | 2.50 g |
| Sodium Lauryl Sulfate | 2.0 mg | 0.20 g |
| Silicon dioxide | 1.0 mg | 0.10 g |
| Total Solid | 210 mg | 21.0 g |

### Example 3

### Capsule Formulations Containing Capsaicin Palmitate

The following ingredients in each one of the capsule formulations were weighed accurately, ground using a pestle and mortar to fine and homogeneous powders. These powders were sieved through 100 mesh and filled into hard gelatin capsules. The composition of each capsule formulation is listed below.

CAPSULE FORMULATION 1

| | In each | In 100 |
|---|---|---|
| Tramadol Hydrochloride | 39.8 mg | 3.98 g |
| Dextromethorphan Hydrochloride | 51.0 mg | 5.10 g |
| Capsaicin Palmitate | 5.4 mg | 0.54 g |
| Ascorbyl Palmitate | 20.0 mg | 2.00 g |
| Microcrystalline Cellulose | 90.8 mg | 9.08 g |
| Sodium Lauryl Sulfate | 1.5 mg | 0.15 g |
| Silicon dioxide | 1.5 mg | 0.15 g |
| Total Solid | 210 mg | 21.0 g |

CAPSULE FORMULATION 2

| | In each | In 100 |
|---|---|---|
| Tramadol Hydrochloride | 39.8 mg | 3.98 g |
| Dextromethorphan Hydrochloride | 51.0 mg | 5.10 g |
| Capsaicin Palmitate | 10.8 mg | 1.08 g |
| Ascorbyl Palmitate | 20.0 mg | 2.00 g |
| Microcrystalline Cellulose | 85.4 mg | 8.54 g |
| Sodium Lauryl Sulfate | 1.5 mg | 0.15 g |
| Silicon dioxide | 1.5 mg | 0.15 g |
| Total Solid | 210 mg | 21.0 g |

CAPSULE FORMULATION 3

| | In each | In 100 |
|---|---|---|
| Tramadol Hydrochloride | 56.9 mg | 5.69 g |
| Dextromethorphan Hydrochloride | 51.0 mg | 5.10 g |
| Capsaicin Palmitate | 10.8 mg | 1.08 g |
| Ascorbyl Palmitate | 25.0 mg | 2.50 g |
| Starch | 38.3 mg | 3.83 g |
| Lactose | 25.0 mg | 2.50 g |
| Sodium Lauryl Sulfate | 2.0 mg | 0.20 g |
| Silicon dioxide | 1.0 mg | 0.10 g |
| Total Solid | 210 mg | 21.0 g |

CAPSULE FORMULATION 4

| | In each | In 100 |
|---|---|---|
| Tramadol Hydrochloride | 56.9 mg | 5.69 g |
| Dextromethorphan Hydrochloride | 51.0 mg | 5.10 g |
| Capsaicin Palmitate | 5.40 mg | 0.54 g |
| Ascorbyl Palmitate | 25.0 mg | 2.50 g |
| Starch | 43.7 mg | 4.37 g |
| Lactose | 25.0 mg | 2.50 g |
| Sodium Lauryl Sulfate | 2.0 mg | 0.20 g |
| Silicon dioxide | 1.0 mg | 0.10 g |
| Total Solid | 210 mg | 21.0 g |

### Example 4

### Capsule Formulations Containing Sildenafil

The following ingredients in each one of the capsule formulations were weighed accurately, ground using a pestle and mortar to fine and homogeneous powders. These powders were sieved through 100 mesh and filled into hard gelatin capsules. The composition of each capsule formulation is listed below.

CAPSULE FORMULATION 1

| | In each | In 100 |
|---|---|---|
| Tramadol Hydrochloride | 39.8 mg | 3.98 g |
| Dextromethorphan Hydrochloride | 51.0 mg | 5.10 g |
| Sildenafil Citrate | 35.2 mg | 3.52 g |
| Microcrystalline Cellulose | 81.0 mg | 8.10 g |
| Sodium Lauryl Sulfate | 1.5 mg | 0.15 g |
| Silicon dioxide | 1.5 mg | 0.15 g |
| Total Solid | 210 mg | 21.0 g |

CAPSULE FORMULATION 2

| | In each | In 100 |
|---|---|---|
| Tramadol Hydrochloride | 39.8 mg | 3.98 g |
| Dextromethorphan Hydrochloride | 51.0 mg | 5.10 g |
| Sildenafil Citrate | 35.2 mg | 3.52 g |
| Starch | 56.0 mg | 5.60 g |
| Lactose | 25.0 mg | 2.50 g |
| Sodium Lauryl Sulfate | 2.0 mg | 0.20 g |
| Silicon dioxide | 1.0 mg | 0.10 g |
| Total Solid | 210 mg | 21.0 g |

CAPSULE FORMULATION 3

| | In each | In 100 |
|---|---|---|
| Tramadol Hydrochloride | 56.9 mg | 5.69 g |
| Dextromethorphan Hydrochloride | 51.0 mg | 5.10 g |
| Sildenafil Citrate | 35.2 mg | 3.52 g |
| Starch | 38.9 mg | 3.89 g |
| Lactose | 25.0 mg | 2.50 g |
| Sodium Lauryl Sulfate | 2.0 mg | 0.20 g |
| Silicon dioxide | 1.0 mg | 0.10 g |
| Total Solid | 210 mg | 21.0 g |

CAPSULE FORMULATION 4

| | In each | In 100 |
|---|---|---|
| Tramadol Hydrochloride | 39.8 mg | 3.98 g |
| Dextromethorphan Hydrochloride | 51.0 mg | 5.10 g |
| Sildenafil Citrate | 49.2 mg | 4.92 g |
| Starch | 42.0 mg | 4.20 g |
| Lactose | 25.0 mg | 2.50 g |
| Sodium Lauryl Sulfate | 2.0 mg | 0.20 g |
| Silicon dioxide | 1.0 mg | 0.10 g |
| Total Solid | 210 mg | 21.0 g |

### Example 5

### Capsule Formulations Containing Sildenafil and Capsaicin Palmitate

The following ingredients in each one of the capsule formulations were weighed accurately, ground using a pestle and mortar to fine and homogeneous powders. These powders were sieved through 100 mesh and filled into hard gelatin capsules. The composition of each capsule formulation is listed below.

CAPSULE FORMULATION 1

| | In each | In 100 |
|---|---|---|
| Tramadol Hydrochloride | 39.8 mg | 3.98 g |
| Dextromethorphan Hydrochloride | 51.0 mg | 5.10 g |
| Capsaicin Palmitate | 5.40 mg | 0.54 g |
| Sildenafil Citrate | 35.2 mg | 3.52 g |
| Microcrystalline Cellulose | 75.6 mg | 7.56 g |
| Sodium Lauryl Sulfate | 1.5 mg | 0.15 g |
| Silicon dioxide | 1.5 mg | 0.15 g |
| Total Solid | 210 mg | 21.0 g |

CAPSULE FORMULATION 2

| | In each | In 100 |
|---|---|---|
| Tramadol Hydrochloride | 39.8 mg | 3.98 g |
| Dextromethorphan Hydrochloride | 51.0 mg | 5.10 g |
| Capsaicin Palmitate | 10.8 mg | 1.08 g |
| Sildenafil Citrate | 35.2 mg | 3.52 g |
| Starch | 45.2 mg | 4.52 g |
| Lactose | 25.0 mg | 2.50 g |
| Sodium Lauryl Sulfate | 2.0 mg | 0.20 g |
| Silicon dioxide | 1.0 mg | 0.10 g |
| Total Solid | 210 mg | 21.0 g |

CAPSULE FORMULATION 3

| | In each | In 100 |
|---|---|---|
| Tramadol Hydrochloride | 56.9 mg | 5.69 g |
| Dextromethorphan Hydrochloride | 51.0 mg | 5.10 g |
| Capsaicin Palmitate | 10.8 mg | 1.08 g |
| Sildenafil Citrate | 35.2 mg | 3.52 g |
| Starch | 28.1 mg | 2.81 g |
| Lactose | 25.0 mg | 2.50 g |
| Sodium Lauryl Sulfate | 2.0 mg | 0.20 g |
| Silicon dioxide | 1.0 mg | 0.10 g |
| Total Solid | 210 mg | 21.0 g |

CAPSULE FORMULATION 4

| | In each | In 100 |
|---|---|---|
| Tramadol Hydrochloride | 39.8 mg | 3.98 g |
| Dextromethorphan Hydrochloride | 51.0 mg | 5.10 g |
| Capsaicin Palmitate | 5.40 mg | 0.56 g |
| Sildenafil Citrate | 49.2 mg | 4.92 g |
| Starch | 36.6 mg | 3.66 g |
| Lactose | 25.0 mg | 2.50 g |
| Sodium Lauryl Sulfate | 2.0 mg | 0.20 g |
| Silicon dioxide | 1.0 mg | 0.10 g |
| Total Solid | 210 mg | 21.0 g |

### Example 6

The subject was a 23 year old white male in good health. On one night, upon arriving at his girlfriend house, the subject consumed one capsule of the test article, formulation 1 in example 4 and then proceeded to drink beer (12 oz beverage, less than 6% alcoholic content). After approximately 1.5 hours since arriving, the subject had also consumed almost 4 beers. The subject became worried about the detrimental effect the alcohol might have on his performance due to the large quantity he had ingested, but these fears were quickly allayed upon commencement of intercourse. The subject was immediately aware of a stronger than expected erection, which helped to quickly dispel any thoughts of performance anxiety, and during the passage of time was equally impressed by the stamina afforded him considering his heavily intoxicated state. Afterwards, the subject could not cite any noticeable negative effects, especially in relation to the combination of the test article with alcohol, which has been shown to have adverse effects with many other classes of drugs and medications.

### Example 7

The subject was 22 yrs old while male and ingested said capsule, formulation 1 in example 4. He noticed a “higher than normal” feeling of arousal at about 1 hour afterwards. He commenced intercourse after 2 hours of taking the capsule, and noticed an increased sexual drive. He was able to sustain an erection for longer and lasted longer (60-75 minutes) before reaching a more delightful orgasm than normal. On second time, he ingested said capsule. This time the subject noticed a greater stiffness in his erection and a comparable experience to before. The length of the intercourse was much longer (55-65 minutes) and his orgasm was more powerful than the previous.

### Example 8

A hispanic male of 63 years old who was a manager in a healthcare company and retired in Mexico. He used to date couple of female partners every week and noticed that during sexual activities with his partners he could not provide sexual satisfaction due to his age. He was provided with capsules of formulation 1 in example 4 and formulation 1 in example 5 and advised to take 1 capsule approximately 2 hours before intercourse and 1 capsule approximately 1 hour before intercourse. He took 2 capsules, formulation 1 in example 4, in the first night and according to his testimony, he felt slightly numb in his penis and he was able to arouse his female partner's sexual feelings by performing pre-intercourse sexual conducts and his partner was able to perform pre-intercourse sexual conducts with his penis for almost half hour without any ejaculation. He was able to perform intercourse for more than 40 minutes and his partner felt exhaustion. His female partner was so ecstatic and he was able to perform sexual intercourse 2 times that night. He is periodically taking the capsules whenever he wants to have a good and sound sexual intercourse for his otherwise stressful body.

### Example 9

The subject was a 45 yrs old businessman and lives in Mexico. He was having problem with ejaculation and was keen on trying the capsule of formulation 1 in example 5. He went for a vacation on a cruise in Europe and used 1 capsule before 2 hours before sexual activities. He called the next day to tell that the capsule worked. It took almost an hour before he could ejaculate. He and his wife were so excited that he was using the capsules several times during the trip for complete sexual satisfaction.

### Example 10

A 44 year old Hispanic male had premature ejaculation problem and wanted to try the formula of the present invention. He was provided with capsules of formulation 1 in Example 3. He took one capsule approximately 2 hours before sexual activities and he gave the following testimony. “The product works so well as I was exhausted doing sex. I have never seen any product like this before”.

### Example 11

A 35 year old Hispanic male wanted to experiment the formula of the present invention for his sexual activities. He was provided with capsules of formulation 1 in Example 4. He took one capsule approximately 2 hours before sexual activities and he gave the following testimony. “I took only one capsule about 2-3 hours before doing my sexual activities. I was doing sex for almost 3-4 hours with intermittent rest as I was getting tired. I wanted to have ejaculation so that I can finish my sexual orgasm. I could not ejaculate the whole night and my partner nearly passed out of our activities. I have never seen any product like this before”.

### Example 12

### The Effect of Sildenafil, Dextromethorphan and Tramadol on CLASS II Males

In order to demonstrate the efficacy of the inventive composition to treat premature ejaculation on CLASS II males, about 30 volunteers have been chosen in Central America from the age groups of 40 and 65 who had premature ejaculation problems. The volunteers were given capsules of formulation 1 in example 4. The volunteers were asked to take 1 or 2 capsules 2-3 hours before the sexual act and were asked to fill out the sexual satisfaction before and after the sexual acts. The results were compiled and analyzed for sexual satisfaction. The results show that more than 90% of the volunteers were extremely satisfied with the composition of the invention for pre-mature ejaculation problems.

### REFERENCES

The following references, to the extent that they provide exemplary procedural or other details supplementary to those set forth herein, are specifically incorporated herein by reference.
1. Albers, G W et al, “Safety and tolerance of oral dextromethorphan in patients at risk for brain ischemia,” Stroke 22: 1075-1077 (1991).
2. Applebaum, J S et al, “Dextromethorphan in the treatment of ALS: A pilot study,” Abstract number 960S (page 393) in Neurology 41 (Suppl, 1), March 1991
3. Balon, “Antidepressants in the Treatment of Premature Ejaculation,” Journal of Sex & Marital Therapy, 22(2):85-96 (1996).
4. Brinn R et al, “Sparteine oxidation is practically abolished in quinidine-treated patients,” Br. J. Clin. Pharmacol. 22: 194-197 (1986).
5. Broly F et al, “Effect of quinidine on the dextromethorphan O-methylase activity of microsomal fractions from human liver,” Br. J. Clin. Pharmacol. 28: 29-36 (1989).
6. Broly F et al, “Inhibitory studies of mexiletine and dextromethorphan oxidation in human liver microsomes,” Biochem. Pharmacol. 39: 1045-1053 (1990).
7. Brosen K et al, “Extensive metabolizers of debrisoquin become poor metabolizers during quinidine treatment,” Pharmacol. Toxicol. 60: 312-314 (1987)
8. Carpenter, C L et al, “Dextromethorphan and dextrorphan as calcium channel antagonists,” Brain Research 439: 372-375 (1988)
9. Cavallini (1995) “Alpha-1 Blockade Pharmacotherapy in Primitive Psychogenic Premature Ejaculation Resistant to Psychotherapy,” Eur. Urology 28:126-130.
10. Choi D W, “Dextrorphan and dextromethorphan attenuate glutamate neurotoxicity,” Brain Res. 402: 333-336 (1987)
11. Craviso G L and Musacchio J M, “High affinity dextromethorphan binding sites in guinea pig brain,” Mol. Pharmacol. 23: 619-640 (1983).
12. David J E et al, “Identification of 6′,7′-Dihydroxybergamottin, a Cytochrome P450 Inhibitor, In Grapefruit Juice”, Drug Metabolisms and Disposition, vol. 24, No. 12, pp. 1287-1290 (1996).
13. Dayer R et al, “Dextromethorphan O-demethylation in liver microsomes . . . ” Clin. Pharmacol. Ther. 45: 34-40 (1989)
14. Feeser et al, Neurosci. Letters 86: 340-345 (1988)
15. Di Silverio et al (1996), “Effects Compares de l′Incision Cervico-Prostatique (ICP) et de l′Association ICP et Agonistes de la LHRH dans le Traitement de l′Hypertrophie Benigne de la Prostate,” Journal D′Urologic 102(3):111-116.
16. Falaschi et al (1981), “Brain Dopamine and Premature Ejaculation: Results of Treatment with Dopamine Antagonists,” Apomorphine and Other Dopaminomitics 1:117-121.
17. Feinberg (1991), “Clomipramine for Obsessive-Compulsive Disorder,” AFP Clinical Pharmacology 43(5):1735-1738.
18. Ferkany et al, Eur. J. Pharmacol. 151: 151-154 (1988)
19. Ferrari et al (1994), “The Selective D2 Dopamine Receptor Antogonist Eticlopride Counteracts the Ejaculatio Praecox Induced by the Selective D2 Dopamine Agonist SND 919 in the Rat,” Life Sciences 55(14):1155-1162. (8 pages)
20. Forme-Pfister et al, Biochem. Biophys. Res. Communic. 148: 1144-1150 (1987)
21. Frank H B et al, “Synthesis and Biological Evaluation of 6′,7′-Dihydroxybergamottin (6,7-DHB), A Naturally Occurring Inhibitor of Cytochrome P450 3A4”, Biorganic & Medicinal Chemistry Letter, vol. 7, No. 20, pp. 2593-2598, 1997.
22. Guttendorf R J et al, “Simplified phenotyping with dextromethorphan by thin-layer chromatography,” Ther. Drug. Monit. 10: 490-498 (1988).
23. Hull et al (1994), “The Roles of Nitric Oxide in Sexual Function of Male Rats,” Neuropharmacology 33(11):1499-1504.
24. Goldstein et al, “Oral Sildenafil in the Treatment of Erectile Dysfunction,” The New England Journal of Medicine, 338, pp 1397-1404 (1998).
25. Inaba T et al, “In vitro inhibition studies of two isozymes of human liver cytochrome P-450,” Drug Metabolism and Disposition 13: 443-447 (1985)
26. Inaba T et al., “Quinidine: Potent inhibition of sparteine and debrisoquin oxidation in vivo,” Br. J. Clin. Pharmacol. 22: 199-200 (1986)
27. Inaba T et al, “Quinidine: Potent inhibition of sparteine and debrisoquin oxidation in vivo,” Br. J. Clin. Pharmacol. 22: 199-200 (1986).
28. Rashid J et al, “Quercetin, an in vitro inhibitor of CYP3A, does not contribute to the interaction between nifedipine and grapefruit juice”, Br J clin Pharmac, vol. 36, pp. 460-463, 1993.
29. Jachau M R “Substrates, Specificities and Functions of the P450 Cytochromes”, LIFE SCIENCES, Vol. 47, pp. 2385-2394 (1990).
30. Neal J J et al., “Inhibition of Insect Cytochromes P450 by Furanocoumarins”, 1994, Pesticide Biochemistry and Physiology 50, pp. 43-50.
31. Jerzy Klinger (2000) “Vita Sexualis:The truth about human sex life”, By Klinger, Pawel, Translated from Polish to English by Klinger, Jerzy, Copyright© 1994 pages 1-362.
32. Kan He et al, “Inactivation of Cytochrome P450 3A4 by Bergamottin, a Component of Grapefruit Juice”, Chem. Res. Toxicol, vol. 11, pp. 252-259, 1998.
33. Katsuyuki Fukuda, et al, “Grapefruit Component Interacting with Rat and Human P450 CYP3A: Possible Involvement of Non-Flavenoid Components in Drug Interaction”, Biol. Pharm. Bull., vol. 20, No. 5, pp. 560-564, May 1997.
34. Koppel C et al., “Urinary metabolism of dextromethorphan in man,” Arzneim.-Forsch./Drug Research 37: 1304-1306 (1987).
35. Koyuncuoglu & Saydam, Intnl. J. Clin. Pharmacol. Ther. Tox. 28: 147-152 (1990)
36. Kupfer A et al “Dextromethorphan as a safe probe for debrisoquine hydroxylation polymorphism,” Lancet ii: 517-518 (1984).
37. Leander, Epilepsy Res. 4: 28-33 (1989)
38. Bourian M et al, “Coumarin Derivatives in Grapefruit Juice and Their Interactions with Mammalian Drug Metabolising Enzyme Systems”, Annual Congress on Medicinal Plant Research, vol. 44, pp. 43, 1996.
39. Obermeier M T et al., “Effects of bioflavonoids on hepatic P450 activities”, Xeonbiotica, vol. 25, No. 6, pp. 575-584, 1995.
40. Marina Tinel, et al., “Inactivation of Human Liver Cytochrome P-450 by the Drug Methoxsalen and Other Psoralen Derivatives”, Biochemical Pharmacology, vol. 36, No. 6, pp. 951-955, 1987.
41. Metz et al (1997), “Premature Ejaculation: A Psychophysiological Review,” Journal of Sex & Marital Therapy 23(1):3-23.
42. Musacchio J M et al, “High affinity dextromethorphan binding sites in the guinea pig brain,” J. Pharmacol. Exp. Ther. 247: 424-431 (1988)
43. Napoli-Farris et al (1984), “Stimulation of Dopamine Autoreceptors Elicits Premature Ejaculation in Rats,” Pharmacology Biochemistry & Behavior 20:69-72.
44. Nielsen M D et al., “A dose-effect study of the in vivo inhibitory effect of quinidine on sparteine oxidation in man,” Br. J. Clin. Pharmacol. 29: 299-304 (1990).
45. Niznik et al, Arch. Biochem. Biophys. 26: 424-432 (1990)
46. Physician's Desk Reference, 44th Edition (1988), pp. 670-671 (Medical Economics Company, 1990).
47. Prince & Feeser, Neurosci. Letters 85: 291-296 (1988).
48. Ramachander G et al, “Determination of dextrorphan in plasma and evaluation of bioavialability dextromethorphan hydrobromide in humans,” J. Pharm. Sci 66: 1047-1048 (1977)
49. Rodd E H, Chemistry of Carbon Compounds (Elsevier Publ., New York, 1960).
50. Steinberg G K et al, “Delayed treatment with dextromethorphan and dextrorphan reduces cerebral damage after transient focal ischemia,” Neurosci Letters 89: 193-197 (1988).
51. Testa B and Jenner P, “Inhibitors Of Cytochrome P-450s and Their Mechanism of Action”, DRUG METABOLISM REVIEWS, 12(1)1-117 (1981); F. P. Guengerich, “Cytochrome P450: Advances and Prospects”, FASEB J., Vol. 6, pp. 667-668 (1992).
52. Tortella et al, TIPS 10: 501-507 (1989)
53. Vettican S J et al, “Phenotypic differences in dextromethorphan metabolism,” Pharmaceut Res. 6: 13-19 (1989)
54. Waldinger et al (1997), “Ejaculation-Retarding Properties of Paroxetine in Patients with Primary Premature Ejaculation: A Double-Blind, Randomized, Dose-Response Study,” British Journal of Urology 79:592-595.
55. Walker E O, and Hunt V P, “An open label trial of dextromethorphan in Huntington's Disease,” Clin. Neuropharmacol. 12: 322-330 (1989).
56. Chan W K et al, “Mechanism-Based Inactivation of Human Cytochrome P450 3A4 by Grapepfruit Juice and Red Wine”, Life Sciences, vol. 62, No. 10, pp. PL 135-142, 1998.
57. Wong B Y et al, “Dextrorphan and dextromethorphan, common antitussives, are antiepileptic and antagonize NMDA in brain slices,” Neurosci Letters 85: 21-26 (1988)
58. Yingna Cai et al, “Inhibition and Inactivation of Murine Hepatic Ethoxy- and Pentoxyresorufin O-Delkylase by Naturally Occurring Coumarins”, Chem. Res. Toxicol, vol. 6, pp. 872-879 (1993).
59. Brosen K, Murray M and Reidy G F, “Recent Developments In Hepatic Drug Oxidation Implications For Clinical Pharmacokinetics”, CLIN. PHARMACOKINET., 18(3): 220-239, 1990.
60. Murray M and Reidy G F, “Selectivity in the Inhibition of Mammalian Cytochrome P-450 By Chemical Agents”, PHARMACOLOGICAL REVIEWS, 42, 85-101 (1990).
61. Porter T D and Coon M J, “Cytochrome P-450: Multiplicity of Isoforms, Substrates, and Catalytic and Regulatory Mechanisms”, J. BIOL. CHEM., Vol. 266, 13469-13472 (1991).
62. Guengerich F P, “Characterization of Human Microsomal Cytochrome P-450 Enzymes”, ANNU. REV. PHARMACOL. TOXICOL. Vol, 29, pp. 241-264 (1989).
63. Martindale, The Extra Pharmacopoeia, 31st edition, pp 333 (London: The Royal Pharmaceutical Society, 1996).
64. Dayer P, Desmeules J, Collart L. Pharmacology of tramadol Drugs 1997; 53 Suppl 2:18-24.
65. Raffa R B. A novel approach to the pharmacology of analgesics. Am J Med 1996; 101(1A):40S-46S.
66. Reimann W, Hennies H H. Inhibition of spinal noradrenaline uptake in rats by the centrally acting analgesic tramadol. Biochem Pharmacol 1994; 47(12):2289-93.
67. Dayer P, Collart L, Desmeules J. The pharmacology of tramadol. Drugs 1994; 47 Suppl 1:3-7.
68. Raffa R B, Friderichs E, Reimann W, Shank R P, Codd E E, Vaught J L, Jacoby H I, Selve N. Complementary and synergistic antinociceptive interaction between the enantiomers of tramadol J Pharmacol Exp Ther 1993; 267: 331-40.
69. Lee C R, McTavish D, Sorkin E M. Tramadol. A preliminary review of its pharmacodynamic and pharmacokinetic properties, and therapeutic potential in acute and chronic pain states. Drugs 1993; 46(2):313-40.
70. Tobias J D. Seizure after overdose of tramadol. South Med J 1997; 90(8):826-7.
71. Weinbroum A A, Valery R, Gideon P, Ben-Abraham R. The role of dextromethorphan in pain control CAN J ANESTH 2000; 47: 585-596.
72. Rodd E H. Chemistry of Carbon Compounds, Elsevier Publ, New York, 1960.
73. Grond S, Thomas M, Detlev Z et al, “Analgesic efficacy and safety of tramadol enantiomers in comparison with the racemate: a randomised, double-blind study with gynaecological patients using intravenous patient-controlled analgesia” Pain 1995; 62(3):313-320.
74. Shipton E A. “Reviews: Tramadol-Present and Future” Anaesth Intensive Care 2000; 28:363-374.
75. Barnung S K, Treschow M, Borgbjerg F M. “Respiratory depression following oral tramadol in a patient with impaired renal function” Pain 1997; 71:111-112.
76. Wiebalck A et al, “Sind Tramadol-Enantiomere für die postoperative Schmerztherapie besser geeignet als das Racemat? Eine randomisierte, Plazebo- and Morphin-kontrollierte Doppelblindstudie”, Der Anaesthesist, 1998; 47: 387-394.
77. Lintz et al., Arzneim.-Forsch./Drug Res. 1981; 31(11), 1932-1943.
78. Rajfer J, Aronson W J, Bush P A, Dorey F J, Ignarro L J. Nitric oxide as a mediator of relaxation of the corpus cavernosum in response to nonadrenergic, noncholinergic neurotransmission. N. Eng. J. Med. 1992; 326: 90-4.
79. Andersson K E, Wagner G. Physiology of the penile erection. Physiol. Rev. 1995; 75: 191-236.
80. Burnett A L. The role of nitric oxide in the physiology of an erection. Biol. Reprod. 1995; 52: 485-9.
81. Boolell M, Allen M J, Ballard S A, Gepi-Attee S, Muirhead G J, Naylor A M, Osterloh I H, Gingell J C. Sildenafil: an orally active type 5 GMP-specific phosphodiesterase inhibitor of penile erection dysfunction. Int. J. Impot. Res. 1996; 8: 47-52.
82. Holzer P (1988). Local effector functions of capsaicin-sensitive sensory nerve endings: involvement of tachykinins, calcitonin gene-related peptide and other neuropeptides. Neuroscience 24: 739-768.
83. Holzer P (1991). Capsaicin: cellular targets, mechanisms of action, and selectivity for thin sensory neurons. Pharmacol Rev 43: 143-201.
84. Winter J, Bevan S and Campbell E A (1995). Capsaicin and pain mechanisms. Br J Anaesth 75: 157-168.
85. Caterina M J and Julius D (1999). Sense and specificity: a molecular identity for nociceptors. Curr Opin Neurobiol 9: 525-530.
86. Caterina M J and Julius D (2001). The vanilloid receptor: a molecular gateway to the pain pathway. Annu Rev Neurosci 24: 487-517.
87. Caterina M J, Schumacher M A, Toming a M, Rosen T A, Levine J D and Julius D (1997). The capsaicin receptor: a heat-activated ion channel in the pain pathway. Nature (Lond) 389: 816-824.
88. Tominaga M, Caterina M J, Malmberg A B, Rosen T A, Gilbert H, Skinner K, Raumann B E, Basbaum A I and Julius D (1998). The cloned capsaicin receptor integrates multiple pain-producing stimuli. Neuron 21: 531-543.
89. Wood J N and Docherty R (1997). Chemical activators of sensory neurons. Annu Rev Physiol 59: 457-482.
90. Kenins P (1982). Response of single nerve fibres to capsaicin applied to the skin. Neurosci Lett 29: 83-88.
91. Lynn B, Ye W and Costell B (1992). The actions of capsaicin applied topically to the skin of the rat on C-fibre afferents, antidromic vasodilation and substance P levels. Br J Pharmacol 107: 400-406.
92. Carter R B and Francis W R (1991). Capsaicin desensitization to plasma extravasation by antidromic C-fibre stimulation is not associated with antinociception in the rat. Neurosci Lett 127: 49-52.
93. McMahon S B, Lewin G and Bloom S R (1991). The consequences of long-term topical capsaicin application in the rat. Pain 44: 301-310.
94. Simone D A, Baumann T K and LaMotte R H (1989). Dose-dependent pain and mechanical hyperalgesia in humans after intradermal injection of capsaicin. Pain 38: 99-107.
95. Singh S, Natarajan K, Aggarwal B B (1996). Capsaicin (8-methyl-N-vanillyl-6-nonenamide) is a potent inhibitor of nuclear transcription factor-κB activation by diverse agents. J. Immunol. 157:4412.
96. Szallasi A & Blumberg P M (1999). Vanilloid (Capsaicin) receptors and mechanisms. Pharmacol. Rev., 51, 159-212.
97. Tanden R et al (1992). “Topical capsaicin in painful diabetic neuropathy. Effect on sensory function.” Diabetes Care. 15, 8-14.
98. Nolano M, Simone D A, Wendelschafer-Crabb G, Johnson T, Hazen E and Kennedy W R (1999). Topical capsaicin in humans: parallel loss of epidermal nerve fibres and pain sensation. Pain 81: 135-145.
99. Watson C P N (1994). Topical capsaicin as an adjuvant analgesic. J Pain Symptom Manage 9: 425-433.
100. Watanabe A. et al, Efficacy of capsaicin ointment (Zostrix) in the treatment of herpetic pain and post-herpetic neuralgia, Pain Clinic 15:709-713, 1994.
101. Bernstein J E. et al, Topical capsaicin treatment of chronic post-herpetic neuralgia, J. Am. Acad. Dermatol. 21: 265-270, 1989.
102. Watson C P N. et al (1993). A randomized vehicle-controlled trial of topical capsaicin in the treatment of post-herpetic neuralgia, Clin. Ther. 15:510-526.
103. Rains C, Bryson H M (1995). Topical capsaicin. A review of its pharmacological properties and therapeutic potential in post-herpetic neuralgia, diabetic neuropathy and osteoarthritis. Drugs Aging. 7(4):317-28.
104. Watson C P N and Evans R J (1992). The postmastectomy pain syndrome and topical capsaicin: a randomized trial. Pain 51: 375-379.
105. Dini D, Bertelli G, Gozza A and Formo G G (1993). Treatment of the post-mastectomy pain syndrome with topical capsaicin. Pain 54: 223-226.
106. Epstein J B and Marcoe J H (1994). Topical capsaicin for treatment of oral neuropathic pain and trigeminal neuralgia. Oral Surg Oral Med Oral Pathol 77: 135-140.
107. Hersh E V, Pertes R A and Ochs H A (1994). Topical capsaicin—pharmacology and potential role in the treatment of temperomandibular pain. J Clin Dent 5: 54-59.
108. Marks D R, Rapoport A, Padla D, Weeks R, Rosum R, Sheftell F and Arrowsmith F (1993). A double-blind placebo-controlled trial of intranasal capsaicin for cluster headache. Cephalgia 13: 114-116.
109. McCarthy G M and McCarthy D J (1992). Effect of topical capsaicin in the therapy of painful osteoarthritis of the hands. J Rheumatol 19: 604-607.
110. Hautkappe M, Roisen M F, Toledano A, Roth S, Jeffries J A and Ostermeier A M (1998). Review of the effectiveness of capsaicin for painful cutaneous disorders and neural dysfunction. Clin J Pain 14: 97-106.
111. Low P A, Opfer-Gehrking T L, Dyck P J, Litchy W J and O'Brien P C (1995). Double-blind, placebo-controlled study of the application of capsaicin cream in chronic distal painful polyneuropathy. Pain 62: 163-168.
112. Paice J A, Ferrans C E, Lashley F R, Short S, Vizgirda V and Pitrak D (2000). Topical capsaicin in the management of HIV-associated peripheral neuropathy. J Pain Symptom Manage 19: 45-52.
113. Fuchs P N, Pappagallo M and Meyer R A (1999). Topical EMLA^{R} pre-treatment fails to decrease the pain induced by 1% topical capsaicin. Pain 80: 637-642.
114. Fuller R W (1991). Pharmacology of inhaled capsaicin in humans. Respirat. Med. 85 (Supp). A): 31-34.
115. Rumsfield, J A, and West D (1991). Topical capsaicin in dermatological and peripheral pain disorders. DICP, Ann. Pharmacotherap. 25: 381-387.
116. Lynn B. Capsaicin: actions on nociceptive C-fibres and therapeutic potential. Pain 1990; 41:61-9.
117. Marsh S J, Stansfeld C E, Brown D A, Davey R, McCarthy D. The mechanism of action of capsaicin on sensory C-type neurons and their axons in vitro. Neuroscience 1987; 23:275-89.
118. Nolano M, Simone D A, Wendclschafer-Crabb G, Johnson T, Hazen E, Kennedy W R. Topical capsaicin in humans: parallel loss of epidermal nerve fibers and pain sensation. Pain 1999; 81:135-45.
119. Van Rijswijk J B, Boeke E L, Keizer J M, Mulder P G, Blom H M, Fokkens W J. Intranasal capsaicin reduces nasal hyperreactivity in idiopathic rhinitis: a double-blind randomized application regimen study. Allergy. 2003 58(8):754-61.
120. Mori A, et al Capsaicin, a component of red peppers, inhibits the growth of androgen-independent, p-53 mutant prostate cancer cells. Cancer Res 2006 66(6):3222-29.
121. Keret D, Goldin E. Topical capsaicin—a novel and effective treatment for idiopathic intractable pruritus ani: a randomised, placebo controlled, crossover study. Gut. 2003; 52(9):1323-6.
122. Abdel-Hamid I A, El Naggar E A, El Gilany A H. Assessment of as needed use of pharmacotherapy and the pause-squeeze technique in premature ejaculation. Int J Impot Res 2001; 13: 41-5.
123. Hull E M, Lumley L A, Matuszewich L, Dominguez J, Moses J, Lorrain D S. The roles of nitric oxide in sexual function of male rats. Neuropharmacology 1994; 33: 1499-504.
124. Kriegsfeld L J, Demas G E, Huang P L, Burnett A L, Nelson R J. Ejaculatory abnormalities in mice lacking the gene for endothelial nitric oxide synthase (eNOS−/−). Physiol Behav 1999; 67: 561-6.
125. Heuer O, Uckert S, Machtens S A, et al. Effects of various nitric oxide donating agents on the contractility and cyclic nucleotide turnover of human seminal vesicles in vitro. Urology 2002; 59: 958-62.
126. Pfaus J G. Neurobiology of sexual behavior. Curr Opin Neurobiol 1999; 9: 751-758.
127. Goldstein I, Lue T F, Padma-Nathan H, Rosen R C, Steers W D, Wicker P A. Oral sildenafil in the treatment of erectile dysfunction. Sildenafil Study Group. N Engl J Med 1998; 338: 1397-404.
128. McMahon, C. G., Samali, R. and Johnson, H.: Efficacy, safety and patient acceptance of sildenafil citrate as treatment for erectile dysfunction. J Urol, 164: 1192, 2000.
129. Padma-Nathan H, Shabsigh R. Sildenafil citrate (Viagra): a review. AUA update series 1999; 18: 274-280.
130. Abdel-Hamid A. Phosphodiesterase 5 inhibitors in rapid ejaculation: Potential use and possible mechanisms of action. Drugs 2004; 64: 13-26.
131. Salonia A, Maga T, Colombo R, et al. A prospective study comparing paroxetine alone versus paroxetine plus sildenafil in patients with premature ejaculation. J Urol 2002; 168: 2486-2489.
132. Safarinejad M R and Hosseini S Y. Pharmacotherapy for Premature Ejaculation, Current Drug Therapy, 2006, 1, 37-46.
133. Markowitz J S, Patrick K S. Venlafaxine-tramadol similarities. Medical Hypotheses 1998; 51:167-8.
134. Lang E, Hord A H, Denson D. Venlafaxine hydrochloride (Effexor) relieves thermal hyperalgesia in rats with an experimental mononeuropathy. Pain 1998; 68:151-5.
135. Schreiber S, Backer M M, Pick C G. The antinociceptive effect of venlafaxine in mice is mediated through opioid and adrenergic mechanisms. Neuroscience Letters 1999; 273: 85-8.
136. Schreiber S, Backer M M, Pick C G. The antinociceptive effect of venlafaxine in mice is mediated through opioid and adrenergic mechanisms. Neuroscience Letters 1999; 273: 85-8.
137. Pantich H S et al., Randomized, Controlled Trial of Dextromethorphan/Quinidine for Pseudo bulbar Affect in Multiple Sclerosis, Ann Neurol 59:780-787, 2006.

1. A method of effectively treating a sexual dysfunction, comprising administering to a patient in need of such treatment an amount of agents including:
a) an NMDA receptor antagonist or a pharmaceutically acceptable salt thereof,
b) tramadol, a derivative or analog of tramadol, or a pharmaceutically acceptable salt thereof, and
c) a capsaicinoid or an esterified capsaicinoid,
wherein the combined amounts of said agents is effective to treat the sexual dysfunction.

2. The method of claim 1, wherein the sexual dysfunction is premature ejaculation.

3. The method of claim 1, wherein said agents are administered separate pharmaceutical preparations.

4. The method of claim 1, wherein said agents are subsequently administered to the patient within a time period of from about 1 second to about 2 hours.

5. The method of claim 1, wherein the agents are administered in a single pharmaceutical composition.

6. The method of claim 1, wherein the pharmaceutical composition is a tablet or capsule.

7. The method of claim 1, wherein the agents are administered prior to sexual activity.

8. The method of claim 1, wherein the agents are administered orally, by means of an implant, parenterally, sub-dermally, sublingually, rectally, topically, or via inhalation.

9. The method of claim 8, wherein the agents are administered orally.

10. The method of claim 1, wherein the NMDA receptor antagonist is dextromethorphan, dextrorphan, ketamine, amantadine, memantine, eliprodil, ifenprodil, phencyclidine, MK-801, dizocilpine, CCPene, flupirtine, or derivatives or salts thereof.

11. The method of claim 10, wherein the NMDA receptor antagonist is dextromethorphan.

12. The method of claim 1, wherein the tramadol or a derivative or analog of tramadol is (1R,2R or 1S,2S)-(dimethylaminomethyl)-1-(3-methoxyphenyl)-cyclohexanol (tramadol), its N-oxide derivative (“tramadol N-oxide”), a O-desmethyl tramadol derivative (“O-desmethyl tramadol”), venlafaxine, (R/S)-1-[2-(dimethylamino)-1-(4-methoxyphenypethyl]cyclohexanol or O-desmethylvenlafaxine.

13. The method of claim 1 wherein said agents are administered in a pharmaceutical composition selected from the group consisting of a tablet, a multiparticulate formulation for oral administration; a solution, a sustained release formulation, a suspension or elixir for oral administration, an injectable formulation, an implantable device, a topical preparation, a solid state and/or depot type transdermal delivery device(s), a suppository, a buccal tablet, or an inhalation formulation such as a controlled release particle formulation or spray, mist, or other topical vehicle intended to be inhaled or instilled into the sinuses.

14. The method of claim 13, wherein the dosage form is further defined as a solid oral dosage form formulated as a tablet or capsule.

15. The method of claim 1, wherein the ratio by weight of NMDA receptor antagonist to the tramadol or the derivative or analog of tramadol is from about 15:1 to 1:15.

16. The method of claim 15, wherein the ratio by weight of NMDA receptor antagonist to the tramadol or the derivative or analog of tramadol is from about 10:1 to 1:10.

17. The method of claim 16, wherein the ratio by weight of NMDA receptor antagonist to the tramadol or the derivative or analog of tramadol is from about 5:1 to 1:5.

18. The method of claim 17, wherein the ratio by weight of NMDA receptor antagonist to the tramadol or the derivative or analog of tramadol is about 1:2.

19. The method of claim 13, wherein the pharmaceutical composition further comprises a phosphodiesterase inhibitor or a pharmaceutically acceptable salt thereof.

20. The method of claim 19, wherein the phosphodiesterase inhibitor is a phosphodiesterase type V inhibitor.

21. The method of claim 19, wherein the phosphodiesterase inhibitor is sildenafil, vardenafil, tadalafil, caffeine, aminophylline, theophylline, aminone, milrinone, vesnarinone, vinpocetine, pemobendan, cilostamide, enoximone, peroximone, rolipram, R020-1724, zaniprast, dipyridamole, MY5445, or IC-351, pyrazolopyrimidinones, polycyclic xanthine derivatives, xanthine derivatives, fused pyridazineanthranilic acid derivatives, fused pyridopyridazinequinazolinone derivatives, quinoline derivatives, thienopyrimidines derivatives, imidazopyridinone derivatives, nitrosated and nitrosylated compounds, aminothiophenecarboxamides or pharmaceutically acceptable salts thereof.

22. The method of claim 19, wherein the ratio by weight of NMDA receptor antagonist to phosphodiesterase inhibitor to the tramadol or the derivative or analog of tramadol is from about 90:1:1 to 1:90:1 to 1:1:90.

23. The method of claim 19, wherein the ratio by weight of NMDA receptor antagonist to phosphodiesterase inhibitor to the tramadol or the derivative or analog of tramadol is from about 5:1:1 to 1:5:1 to 1:1:5.

24. The method of claim 19, wherein the ratio by weight of NMDA receptor antagonist to capsaicinoid, capsaicin, or ester of capsaicin to the tramadol or the derivative or analog of tramadol is from about 90:1:1 to 1:90:1 to 1:1:90.

25. The method of claim 13, wherein the pharmaceutical composition comprises a capsaicinoid selected from the group consisting of capsaicin, capsaicin palmitate, civamide, homocapsaicin, nordihydrocapsaicin, dihydrocapsaicin, homodihydrocapsaicin, n-vanillyloctanamide, nonivamide and n-vanillyldecanamide.

26. The method of claim 13, wherein the esterified capsaicin is of formula (I):
R—CO—CAP (I)
wherein CAP is capsaicin, a capsaicin analogue, civamide, homocapsaicin, nordihydrocapsaicin, dihydrocapsaicin, homodihydrocapsaicin, n-vanillyloctanamide, nonivamide or n-vanillyldecanamide; wherein R is a C₁-C₁₈ alkyl group, a C₁-C₁₈ aryl group, a C₁-C₁₈ alkylene group, a C₁-C₁₈ arylene group, —CH2-CH2-COOH or a c-pentenyl group.

27. The method of claim 26, wherein R is selected from the group consisting of methyl, ethyl, propyl, butyl, hexyl, heptyl, octyl, dodecyl, 1-pentadecyl, 1-heptadecyl, isopropyl, sec-butyl, t-butyl, 2-methylbutyl, 2-pentyl, 3-pentyl, cyclopropyl, cyclobutyl, cyclopentyl and cyclohexyl, vinyl (ethenyl), 1-propenyl, i-butenyl, pentenyl, hexenyl, n-decenyl, —CH2-CH2-COOH and c-pentenyl groups.

28. The method of claim 25, wherein the pharmaceutical composition comprises capsaicin.

29. The method of claim 25, wherein the pharmaceutical composition comprises capsaicin palmitate.

30. A pharmaceutical composition for the treatment of premature ejaculation in humans, comprising a therapeutically effective amount of a combination of agents, the combination comprising:
a) an NMDA receptor antagonist or a pharmaceutically acceptable salt thereof,
b) tramadol or a derivative or analog of tramadol, or a pharmaceutically acceptable salt thereof, and
c) a capasicinoid or an esterified capsaicinoid.

31. The pharmaceutical composition of claim 30, wherein the composition further comprises a phosphodiesterase type V inhibitor, or a pharmaceutically acceptable salt thereof.

32. The pharmaceutical composition of claim 30, wherein the composition comprises capsaicin.

33. The pharmaceutical preparation of claim 30, wherein the composition comprises capsaicin palmitate.

34. The pharmaceutical composition of claim 30, wherein the composition further comprises a phosphodiesterase type V inhibitor.

35. The pharmaceutical composition of claim 34, wherein phosphodiesterase type V inhibitor is sildenafil.
